(19) United States
(12) Patent Application Publication
(10) Pub. No.: US 2014/0041064 A1
(43) Pub. Date: Feb. 6, 2014

(54) COMPOSITIONS AND METHODS FOR IDENTIFYING VIRULENCE FACTORS AND ANTI-FUNGAL AGENTS

(71) Applicant: Reeta Prusty Rao, Newton, MA (US)

(72) Inventor: Reeta Prusty Rao, Newton, MA (US)

(73) Assignee: WORCESTER POLYTECHNIC INSTITUTE, Worcester, MA (US)

(21) Appl. No.: 14/003,349

(22) PCT Filed: Mar. 7, 2012
(86) PCT No.: PCT/US12/28116

§ 371 Date: Oct. 25, 2013

Related U.S. Application Data
(60) Provisional application No. 61/449,802, filed on Mar. 7, 2011.

(51) Int. Cl. G01N 33/50

(52) U.S. Cl. CPC G01N 33/5085
USPC 800/8; 424/9.2; 506/10

(57) ABSTRACT

The present invention is based on the discovery of a two- or three-organism model system that can be used to identify virulence factors involved in fungal infection and to screen for agents that attenuate the activity of those factors. The system includes a model eukaryote, the nematode, Caenorhabditis elegans, and one or more species of Candida, a fungal pathogen that infects humans, e.g., Candida albicans, C. dubliniensis, C. krusei, C. tropicalis, C. parapsilosis, or C. glabrata. The system also includes a food source for the C. elegans; the food source can be a live or attenuated bacterium, e.g., Escherichia coli.

### CROSS-REFERENCE TO RELATED APPLICATIONS

This application claims the benefit of the filing date of U.S. Application No. 61/449,802, filed Mar. 7, 2011. For the purpose of any U.S. patent that may issue from the present application, the entire content of U.S. Application No. 61/449,802 is hereby incorporated by reference herein.

### FIELD OF THE INVENTION

The present invention generally relates to multi-organism model systems and methods for using these systems and components thereof to screen for antimicrobial agents, host factors, and virulence factors that impact infections caused by microorganisms such as fungi.

### BACKGROUND

In recent years, the incidence of invasive fungal infections has escalated, primarily in hospital settings (Pfaller, Clin. Infect. Dis. 22 Suppl 2:S89-94, 1996). Ninety percent of these infections are caused by various Candida species (Fridkin et al., Clin. Microbiol. Rev. 9:499-511, 1996), 50% of which are fatal (Gudlaugsson et al., Clin. Infect. Dis. 37:1172-1177, 2003) and the estimated annual cost of treating nosocomial Candida infections exceeds $1 billion per year and has an attributable mortality of about 5,000 deaths per year in the United States (Miller et al., Clin. Infect. Dis. 32:1110, 2001; Morgan et al., Infect. Control. Hosp. Epidemiol. 26:540-547, 2005; Pappas et al., Clin. Infect. Dis. 37:634-643). Candida species are the fourth leading cause of bloodstream infections (BSI), with C. albicans responsible for about half the cases. C. albicans form robust biofilms on medical implants, such as intravenous catheters, prosthetic joints, and artificial heart valves which can seed potentially lethal disseminated infections (Richards et al., Infect. Control. Hosp. Epidemiol. 21:510-515, 2000; Velasco et al., Sao Paulo Med. J. 118:131-138, 2000; and Wisplinghoff et al., Pediatr. Infect. Dis. J. 22:686-691, 2003). Candida species also cause superficial infections on mucosal surfaces in the body including the mouth, upper gastrointestinal (GI) and uro-gential tracts. At least 75% of women have at least one episode of vaginitis caused by Candida in their lifetime (Sobel, Ann. N.Y. Acad. Sci. 544:547-557, 1988) and oropharyngeal thrush and esophagitis are common in both infants and in patients with AIDS (Darouiche, Clin. Infect. Dis. 26:259-272; quiz 273-254, 1998). C. albicans accounts for the vast majority of mucosal infections. The frequency of these superficial infections combined with the treatment challenges posed by disseminated infections make C. albicans an important pathogen for further study.

The development of antifungal agents has lagged behind that of antibacterial agents. Anti-infective agents typically act by exploiting physiological differences between the infectious organism and the host. Because bacteria are prokaryotic, they offer numerous targets that differ from those of the human host. In contrast, fungal and mammalian cells are both eukaryotes, so that most agents that are toxic to fungi are also toxic to the host. Furthermore, because fungi generally grow slowly and often in multicellular forms, they are more difficult to quantify than bacteria. There is a continuing need for new antifungal agents and systems designed to discover them.

### SUMMARY

The present invention features, inter alia, tools for studying the pathology of conditions caused by microorganisms, such as mucosal candidiasis. The tools include multi-organism model systems, kits, related components, methods for identifying targets for antimicrobial agents (including anti-fungal agents) and methods for identifying such antimicrobial agents per se. An assay to screen for fungal virulence factors has been developed (see Jain et al., Eukaryot. Cell. 8:1218-1227, 2009), and we have validated the screen for pathogenic clinical isolates (see Table 1). Our virulence assays can be differentiated from others that use C. elegans because, first, they expose the nematode host to a small amount of the pathogen (e.g., C. albicans) as a mixed culture with the food source (e.g., E. coli). Other studies generally feed the pathogen only, which does not mimic an infection situation accurately because the host rarely encounters the pathogen as a pure culture. In addition, using a bacterium such as E. coli enables us to conduct genomic mutant screens on the host by using the bacterium to introduce an RNAi knockdown collection. This collection is available in plasmids where E. coli has been modified to express double stranded RNA that is specific to certain C. elegans genes, which knock down the gene's encoded protein concentration to a negligible amount. Second, the present model systems allow us to monitor several aspects of disease progression, namely, intestinal distension, swelling in the vulva and anal regions and death, versus other assays that monitor just death. This enables us to identify fungal virulence factors that affect all stages of the disease process (or a particular stage). Third, our assay is amenable to high throughput screens because it does not involve handling individual animals. The process can be automated using a liquid handling robot because we use suspensions of worm eggs and liquid cultures of the pathogen.

Accordingly, in one aspect, the present invention features a multi-organism model system that includes, in co-culture, the nematode Caenorhabditis elegans (C. elegans), a fungal pathogen that infects humans, and a source of food for the C. elegans. We refer to the circumstance in which the organisms are brought together as a co-culture to indicate that the organisms are brought together under conditions (e.g., temperature and pH) favorable to their survival and observation. The C. elegans can be a wild type C. elegans and the pathogen can include a mutant gene. In some embodiments, the wild type correlate of the mutant gene encodes a protein that increases the pathogen's resistance to reactive oxygen species. Alternatively, the C. elegans can be a mutant C. elegans and the pathogen can be a wild type pathogen. In some embodiments, the wild type correlate of the gene that is mutant in the mutant C. elegans will encode a protein that facilitates the production of reactive oxygen species in C. elegans.

The food source can vary. However, we have discovered that it can be important to provide a food source for the C. elegans, including a source that is ingestible by smaller, younger worms, in order to more accurately assay the impact of a given pathogen on the worms and to more accurately reflect a natural environment where food is available. In any of the model systems described herein, the source of the food can be a heterologous cell (i.e., a cell different from nematode cells and different from the pathogen present in the model system). For example, the heterologous cell can be a bacterial cell, and the cell can be optionally engineered to express a potential anti-fungal agent or an agent that inhibits gene expression in C. elegans. As the nematode consumes the heterologous cell, cells that have been genetically engineered serve two purposes. The first purpose is nourishment to the host organism and the second is as a delivery vehicle of another agent (i.e., a potential anti-fungal agent or an agent that inhibits gene expression in C. elegans).

The co-cultures can be configured in a multi-well plate and/or configured for high throughput screening.

As noted, the multi-organism model systems described herein can include a potential antifungal agent, which may be simply added to the system or delivered by bacterial expression as described above. The potential antifungal agent can be any agent known in the art, including a nucleic acid, polypeptide (a term we use to refer to amino acid polymers, regardless of length), a PNA, a lipid or lipid-based agent, or an organic molecule (e.g., a small organic compound as found, for example, in a compound library).

In the model systems, the fungal pathogen can be of the genus Candida, but the invention is not so limited. Fungi of the genus Saccharomyces are specifically excluded. For example, the fungal pathogen can be C. albicans, C. ascalaphidarum, C. amphixiae, C. antarctica, C. argentea, C. atlantica, C. atmosphaerica, C. blattae, C. carpophila, C. carvajalis, C. cerambycidarum, C. chauliodes, C. corydali, C. dosseyi, C. dubliniensis, C. ergatensis, C. fructus, C. glabrata, C. fermentati, C. guilliermondii, C. haemulonii, C. insectamens, C. insectorum, C. intermedia, C. jeffresii, C. kefyr, C. krusei, C. lusitaniae, C. lyxosophila, C. maltosa, C. marina, C. membranifaciens, C. milleri, C. oleophila, C. oregonensis, C. parapsilosis, C. quercitrusa, C. rugosa, C. sake, C. shehatea, C. temnochilae, C. tenuis, C. theae, C. tropicalis, C. tsuchiyae, C. sinolaborantium, C. sojae, C. subhashii, C. viswanathii, or C. utilis (or any combination thereof).

In the model systems, one can include an agent that inhibits the expression of a gene or the activity of a gene product in the C. elegans or in the human pathogen. These agents include nucleic acids, polypeptides, PNAs, lipids, lipid-based agents, and organic molecules (e.g., a small organic compound as found, for example, in a compound library). The agent can inhibit the expression of a gene or the activity of a gene product.

Based on our work, we expect genes one may wish to mutate in a fungal pathogen include one or more of the genes CMP1, IFF11, SAP8, DOT4, orf19.6713, orf19.1219, and ZCF15 and a homolog or homologs thereof.

In another aspect, the present invention features kits that include (a) instructions for use in identifying a host factor, a virulence factor, or an anti-fungal agent and (b) a multi-organism model system as described herein or one or more components thereof. For example, the component can be the nematode C. elegans, eggs of the nematode C. elegans, a fungal pathogen that infects humans, and/or a source of food for the C. elegans. The C. elegans can be a wild type C. elegans and the pathogen can include a mutant gene. Alternatively, the C. elegans can be a mutant C. elegans and the pathogen can be a wild type pathogen.

In another aspect, the invention features methods of identifying a host factor that promotes fungal pathogenesis. These methods can include the steps of: (a) providing a co-culture including a C. elegans having a mutant gene, a wild type fungal pathogen that infects humans, and, optionally, a source of food for the C. elegans; and (b) carrying out an assay to determine whether the fungal pathogen infects the C. elegans. Inhibition of the expected infection level identifies the protein encoded by the wild type correlate of the mutant gene as a host factor that promotes fungal pathogenesis.

In another aspect, the invention features methods of identifying a fungal virulence factor. These methods can include the steps of: (a) providing a co-culture comprising a wild type C. elegans and a mutant fungal pathogen that, in wild type form, infects humans, and, optionally, a source of food for the C. elegans; and (b) carrying out an assay to determine whether the mutant fungal pathogen infects the C. elegans Inhibition of the expected infection level identifies the protein encoded by the wild type correlate of the mutant gene as a fungal virulence factor.

In another aspect, the invention features methods of identifying a gene in a fungal pathogen that helps protect the pathogen from reactive oxygen species. These methods can include the steps of: (a) providing, in co-culture, a mutant host organism that is deficient in the production of reactive oxygen species and a fungal pathogen comprising a mutation; and (b) carrying out an assay to determine the extent to which the mutant host organism is infected by the fungal pathogen. A degree of infection that is greater than the degree of infection observed when the pathogen is exposed to a wild type host organism indicates that the mutation is in a gene that helps protect the fungal pathogen from reactive oxygen species. The methods can also include (c): exposing the gene that protects the fungal pathogen from reactive oxygen species to a potential anti-fungal agent. These methods can also include step (d): determining whether the potential anti-fungal agent reduces the expression of the gene sequence or inhibits a protein encoded by the gene sequence. An agent that reduces the expression of the gene sequence or inhibits a protein encoded by the gene sequence, is a putative anti-fungal agent. In these methods the gene can be CMP1, IFF11, SAP8, DOT4, orf19.6713, orf19.1219, ZCF15 or a homolog thereof. In any of these methods, one can also include, in the co-culture, a source of food for the C. elegans (e.g., a bacterium, such as E. coli). In any of these methods, the fungal pathogen can be of the genus Candida. Saccharomyces may be explicitly excluded. As noted, the methods of the invention can be carried out where the co-culture is configured in a multi-well plate and/or configured for high throughput screening. When tested, a potential antifungal agent may take many forms, including that of a nucleic acid, polypeptide, or organic molecule. When identifying a gene in a fungal pathogen that helps protect the pathogen from reactive oxygen species, the fungal pathogen can be of the genus Candida (e.g., C. albicans, C. ascalaphidarum, C. amphixiae, C. antarctica, C. argentea, C. atlantica, C. atmosphaerica, C. blattae, C. carpophila, C. carvajalis, C. cerambycidarum, C. chauliodes, C. corydali, C. dosseyi, C. dubliniensis, C. ergatensis, C. fructus, C. glabrata, C. fermentati, C. guilliermondii, C. haemulonii, C. insectamens, C. insectorum, C. intermedia, C. jeffresii, C. kefyr, C. krusei, C. lusitaniae, C. lyxosophila, C. maltosa, C. marina, C. membranifaciens, C. milleri, C. oleophila, C. oregonensis, C. parapsilosis, C. quercitrusa, C. rugosa, C. sake, C. shehatea, C. temnochilae, C. tenuis, C. theae, C. tropicalis, C. tsuchiyae, C. sinolaborantium, C. sojae, C. subhashii, C. viswanathii, or C. utilis. Where the potential anti-fungal agent is a nucleic acid sequence, exposing the gene that protects the fungal pathogen from reactive oxygen species to the potential anti-fungal agent can be accomplished by co-culturing the fungal pathogen and a heterologous cell that expresses the nucleic acid sequence (e.g., a bacterial cell or mammalian cell). The fungal pathogen can include a mutation in one or more of the genes CMP1, IFF11, SAP8, DOT4, orf19.6713, orf19.1219, and ZCF15 or a homolog or homologs thereof.

In another aspect, the invention features methods of generating an impaired fungal pathogen. The methods can be carried out by providing, in co-culture, a fungal pathogen and a heterologous cell that has been genetically engineered to express a nucleic acid sequence that inhibits the expression of a gene in the fungal pathogen. Alternatively, a protein encoded by the nucleic acid sequence may inhibit the activity of the protein encoded by the gene in the fungal pathogen.

In another aspect, the invention features nucleic acids inhibits the expression of the gene CMP1, IFF11, SAP8, DOT4, orf19.6713, orf19.1219, ZCF15 or a homolog of any of these genes.

In another aspect, the invention features an expression vector (e.g., a plasmid, cosmid, or viral vector) that encodes the nucleic acids just described.

In another aspect, the invention features host cells that include the expression vectors just described. The host cells can be bacterial cells, such as E. coli.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 depicts the results of an experiment showing that C. albicans induces deformed anal region (Dar) in wild type worms. Worms were exposed to E. coli as control (Uninfected) and C. albicans (2 examples of infected worms) and pictures were taken on day 4 (Arrow indicates the Dar region). Scale—20 μm.

FIGS. 2A and 2B show different phenotypes due to Candida infection. FIG. 2A shows that exposure to C. albicans causes intestinal distention in the worms over time (day 2, 3 and 4). DIC (Nomarski) pictures of worms feeding on E. coli was taken as control and the DIC, RFP and merged pictures of worms infected with C. albicans are shown over three days. FIG. 2B shows that vulva swelling was observed (arrow points to the vulval region) by day 4 when worms were exposed to Candida compared to the worms exposed E. coli as control (Uninfected). Scale—20 μm.

FIGS. 3A-3D depict the results of an experiment showing that Cap1 was a virulence factor that was required for counteracting oxidative stress created by the host during fungal pathogenesis. Dar phenotype was observed for both wild type (FIG. 3A) and bli-3 mutant worms (FIG. 3B), which lack the ability to produce ROS, exposed to CAP1/CAP1, cap1 Δ/Δ+CAP1 and cap1 Δ/Δ strains (* denotes p<0.001). n denotes the number of worms exposed to a particular strain. Experiment was done in triplicates. FIG. 3C shows survival curves for WT worms when exposed to CAP1/CAP1, cap1 Δ/Δ+CAP1 and cap1 Δ/Δ and E. coli OP50 as control (p<0.01 for CAP1/CAP1 and cap1 Δ/Δ mutant) shows that worms exposed to cap1 Δ/Δ survived longer than worms exposed to wild type. FIG. 3D shows that bli-3 mutant worms exposed to Candida strains showed no difference in survival between exposure to wild type CAP1/CAP1 and cap1 Δ/Δ mutant.

FIG. 4 depicts the results of an experiment showing that Cap1 was required for survival in macrophages. Macrophages were exposed to different strains of Candida in a ratio of 1:15 macrophages with and without DPI. Percentage survival was calculated by dividing the cfu's obtained for Candida grown with macrophages to candida alone. The data was then normalized to the wild type. % survival of cap1 Δ/Δ mutant is significantly less (** p<0.05) than the wild type and the complemented Cap1 strain.

FIG. 5 depicts the results of an analysis showing that Candida virulence was reduced when Cap1 was deleted. 10 mice/strain were injected in the tail vein with 10⁶ cells of respective candida strain. Survival curve of mice when infected with CAP1/CAR1, cap1 Δ/Δ+CAP1 and cap1 Δ/Δ strains shows that mice infected with cap1 Δ/Δ mutant were able to survive significantly longer than those injected with the wild type. (p<0.01 for CAP1/CAP1 and cap1 Δ/Δ mutant).

### DETAILED DESCRIPTION

The present invention is based, in part, on the inventors' discovery of a two organism co-culture system for analysis of fungal pathogenesis. The system can be used with fungal pathogens that typically infect humans. The co-culture system includes a mutant or wild-type nematode, Caenorhabditis elegans, as a model eukaryotic host and one or more species of Candida, e.g., Candida albicans, C. dubliniensis, C. krusei, C. tropicalis, C. parapsilosis, or C. glabrata. The system is useful for identification both host factors that promote fungal pathogenesis and fungal virulence factors that promote and maintain infection. More specifically, the inventors have identified a role for genes involved in the production of reactive oxygen species in fungal infection. The system can be used to identify genes in fungal pathogens that increase the pathogen's resistance to reactive oxygen species. The system can be used to identify genes in the host that facilitates the production of reactive oxygen species in C. elegans. The system is also useful as a screen for anti-fungal agents.

Model Systems:

The multi-organism model system described herein includes a model eukaryotic host and a fungal pathogen. The model host is the nematode, Caenorhabditis elegans. C. elegans is a non-parasitic soil-living nematode that has innate immune responses that defend it against pathogens. Innate immunity functions as a rapid response to infection. Antigen receptors in innate immunity are of limited diversity and specificity. This simple form of immunity is common to all animals. Molecular studies of the pathways and components of innate immunity indicate that they are conserved between invertebrates and vertebrates. C. elegans make a good model for our study because these animals have genes with vertebrate homologs. The worm's hermaphroditic nature and lifespan of two weeks allows for extensive observations. The C. elegans genome has been completely sequenced and mutants are readily available.

The fungal pathogen can be a fungus that infects humans. The fungus can be a laboratory strain or a clinical isolate. Exemplary fungal species include, for example, C. albicans, C. ascalaphidarum, C. amphixiae, C. antarctica, C. argentea, C. atlantica, C. atmosphaerica, C. blattae, C. carpophila, C. carvajalis, C. cerambycidarum, C. chauliodes, C. corydali, C. dosseyi, C. dubliniensis, C. ergatensis, C. fructus, C. glabrata, C. fermentati, C. guilliermondii, C. haemulonii, C. insectamens, C. insectorum, C. intermedia, C. jeffresii, C. kefyr, C. krusei, C. lusitaniae, C. lyxosophila, C. maltosa, C. marina, C. membranifaciens, C. milleri, C. oleophila, C. oregonensis, C. parapsilosis, C. pseudotropicalis, C. quercitrusa, C. rugosa, C. sake, C. shehatea, C. temnochilae, C. tenuis, C. theae, C. tropicalis, C. tsuchiyae, C. sinolaborantium, C. sojae, C. subhashii, C. viswanathii, C. utilis. Aspergilllus spp., Penecillium spp., Stachybotrys spp., Trichoderma spp., Mycoplasma spp., or Histoplasma capsulatum.

The host and the fungal pathogen can be wild type or either can include mutations in one or more genes. The mutations can be in the coding sequence of a gene or in a regulatory region or both. Depending upon the particular sequence, expression of the polypeptide encoded by the gene can be modulated, e.g., increased, decreased or eliminated relative to that of the corresponding wild type gene. Methods of making mutations are well known in the art and include PCR-based method that target specific genes or random mutagenesis using, for example, chemical agents or radiation.

The multi-organism model systems can also include a food source for C. elegans. The food source can be a heterologous cell. The heterologous cell can be a bacterium, typically Escherichia coli, although other species including Bacillis simplex, Bacillis megaterium, Comomonas spp. can also be used. Regardless of the species, the bacteria can be live or attenuated, for example by heat or radiation treatment. Optionally, the heterologous cell can be engineered to express a potential anti-fungal agent or an agent that inhibits gene expression in C. elegans.

Screening:

The model systems provided herein are useful for the identification of genes that mediate infection by fungal pathogens. The genes can be host genes, i.e., a region in the host's genome that encodes sequences that affect the efficiency of infection and the ability of the host to fight the infection. The genes can also be genes expressed by the pathogen, for example, virulence genes. A virulence factor can be any substance produced by the fungal pathogen that is necessary or survival in the host. Some virulence factors alter host-pathogen interaction by increasing the degree of damage done to the host. Virulence factors are used by pathogens in many ways, including, for example, in cell adhesion or colonization of a niche in the host, to evade the host's immune response, to facilitate entry to and egress from host cells, to obtain nutrition from the host, or to inhibit other physiological processes in the host. Virulence factors can include enzymes, endotoxins, adhesion factors, motility factors, factors involved in complement evasion, and factors that promote biofilm formation.

Some virulence factors allow fungal pathogens to resist certain host defenses such as reactive oxygen species (ROS). Reactive oxygen species are a group of chemically reactive ions, radicals and molecules derived from oxygen. Exemplary ROS include hydrogen peroxide H₂O₂, hypochlorite ion ClO⁻, radicals, e.g., the hydroxyl radical, .OH, superoxide ion .O₂⁻.

ROS are play a role in many cellular processes including microbial killing. Phagocytes, e.g., neutrophils, monocytes, macrophages, dendritic cells, and mast cells, exert their anti-microbial effects by engulfing and ingesting harmful microbes, including yeast and bacteria. Phagocytes produce ROS in the phagosome through the activity of the NADPH oxidase complex. Neutrophils also synthesize ROS through myeloperoxidase.

Macrophages and neutrophils generate ROS in order to kill microbes that they engulf by phagocytosis. The process is highly regulated. In brief, bacteria are engulfed into a phagosome, which then fuses with a lysosome. Subunits of the enzyme NADPH oxidase assemble in the lysosome membrane forming the active enzyme. NADPH oxidase catalyzes the synthesis of the superoxide anion, resulting in a large increase in oxygen consumption, called the “respiratory burst”. Superoxide dismutase (SOD) converts this into hydrogen peroxide, which kills the engulfed microbes. Neutrophils, in addition to relying on NADPH oxidase, also kill engulfed pathogens by using the enzyme myeloperoxidase, which catalyzes the reaction of hydrogen peroxide (made from superoxide anions) with chloride ions to produce the strongly antiseptic hypochlorite ion.

Many pathogens, including fungal pathogens such as Candida have mechanisms for preventing the generation of ROS by the host or for avoiding contact with ROS. Through these mechanisms, these pathogenic organisms evade or modulate host immune responses.

The methods described herein are useful for identifying fungal genes that protect the fungal pathogen from ROS. In one embodiment, the methods include providing a co-culture comprising a mutant host organism, e.g., a C. elegans, that is deficient in the production of reactive oxygen species and a fungal pathogen comprising a mutation and determining whether the mutant host organism is infected by the fungal pathogen, wherein a degree of infection that is greater than the degree of infection observed when the pathogen is exposed to a wild type host organism indicates that the mutation is in a gene that protects the fungal pathogen from reactive oxygen species. Various control co-cultures that include different combinations of wild type and mutant host and wild type and mutant fungal pathogens. For example, wild-type and mutant hosts can be co-cultured in the presence of mutant fungal pathogens. Conversely wild-type and mutant hosts can be co-cultured in the presence of mutant fungal pathogens. Fungal pathogens harboring mutations that known to increase susceptibility to ROS, e.g., CAP1, can also be used as controls.

Any method known in the art can be used to assay the host response to infection by fungal pathogens. Useful methods for C. elegans include microscopic analysis of certain phenotypic features, e.g., the DAR phenotype (“deformed anal region”), intestinal distention, and vulval swelling. Alternatively, or in addition, response to infection can be assayed in terms of cell survival rates. Cell death can be assayed by counting the number of dead worms directly or with assays that rely on colorimetric indicator dyes, e.g., 3-(4,5-dimethylthiazol-2-yl)-2,5-diphenyl tetrazolium bromide-formazan (MTT-formazan).

The model system described herein is can also be used to identify agents that increase susceptibility of a fungal pathogen to ROS. Compounds useful in the method are those that kill or substantially inhibit the growth of infectious microorganisms of interest in vitro and/or in vivo. As used herein, “substantial inhibition of growth” means at least two-fold (i.e., at least: three-fold; four-fold; five-fold; six-fold; seven-fold; eight-fold; nine-fold; ten-fold; 25-fold; 50-fold; 100-fold; 1,000-fold; 10,000-fold; 100,000-fold, or even greater) inhibition of growth. In the case of in vivo methods, the killing or substantial inhibition of growth will generally be at a concentration of the inhibitory compound that is not fatally toxic to the host organism.

Agents useful as anti-fungal agents can be identified from libraries (e.g., combinatorial or compound libraries, including those that contain synthetic and/or natural products, and custom analog libraries, which may contain compounds based on a common scaffold). Such libraries can include hundreds or thousands of distinct compounds or random pools thereof. Libraries suitable for screening can be obtained from a variety of sources, including the compound libraries from ChemBridge Corp. (San Diego, Calif.). Another compound library is available from the consortium formed by the University of Kentucky, the University of Cincinnati Genome Research Institute and the Research Institute of the Children's Hospital of Cincinnati. The library is referred to as the UC/GRI Compound Library. The compound libraries employed in this invention may be prepared by methods known in the art. For example, one can prepare and screen compounds that target host or virulence factors by any means including, but not limited to, combinatorial chemistry techniques, fermentation methods, plant and cellular extraction procedures and the like. Methods for making combinatorial libraries are well-known in the art. See, for example, E. R. Felder (Chimia 48:512-541, 1994); Gallop et al. (J. Med. Chem. 37:1233-1251, 1994); R. A. Houghten (Trends Genet. 9:235-239, 1993); Houghten et al. (Nature 354:84-86, 1991); Lam et al. (Nature 354:82-84, 1991); Carell et al. (Chem. Biol. 3:171-183, 1995); Madden et al. (Perspectives in Drug Discovery and Design 2, 269-282); Cwirla et al. (Biochemistry 87:6378-6382, 1990); Brenner et al. (Proc. Natl. Acad. Sci. USA 89:5381-5383, 1992); Gordon et al., (J. Med. Chem. 37:1385-1401, 1994); Lebl et al. (Biopolymers 37:177-198, 1995); and references cited therein.

The assays described herein, e.g., methods of screenign for mutants and methods of screening for compounds that attenuate fungal virulence, can be automated. The assays can be formatted for use so that multiple samples can be screened simultaneously, for example, as a high throughput screen using methods know to those in the art. Typical high throughput screening assays include robotic instrumentation for plating cells into multiwell plates. The multiwell plates can be 96-, 384- or 1536-well plates. For screening of mutants, each assay plate generally includes positive and negative control cells, e.g., various combinations of mutant and wild-type host, plated with various combinations of known mutant and wild type fungal pathogens. For screening of compounds, each assay plate includes both positive and negative control compounds. Concentrated stocks of compounds can be added from stock plates. Typical high throughput screening systems general can analyze between 10,000 and 100,000 compounds per day, but of course, the through put will vary according to the particular assay used for the screen.

Polypeptides:

The term “polypeptide” as used herein refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. The term “amino acid” refers to natural and/or unnatural or synthetic amino acids, including D/L optical isomers. Full-length proteins, analogs, mutants, and fragments thereof are encompassed by this definition.

Polypeptides described herein include Candida polypeptides that function as virulence factors. Virulence factor polypeptides can confer resistance to host ROS. Modulation of the level of virulence factor polypeptides can be either an increase or a decrease in the level of virulence factor polypeptides relative to the corresponding levels in a control fungus.

A virulence factor polypeptide can be encoded by any of the genes listed in Table 4. More specifically, useful virulence factor polypeptides include those shown in Example 10 and encoded by the sequences set forth as SEQ ID NOs.: 1-8. A virulence factor polypeptide can also be a polypeptide that regulates the synthesis of a virulence factor polypeptide, for example, a transcription factor. Transcription factors are a diverse class of proteins that regulate gene expression through specific DNA binding events. Transcription factors are involved in a variety of regulatory networks of genes in fungal pathogens, including those genes responsible for the biosynthesis of metabolites. Transcription factors include a number of characteristic structural motifs that mediate interactions with nucleic acids.

A virulence factor polypeptide can be encoded by any of the sequences set forth in SEQ ID NOs: 1-8. Alternatively, a virulence factor polypeptide can be a homolog, ortholog, or variant of a polypeptide having an amino acid sequence encoded by SEQ ID NOs: 1-8. For example, a virulence factor polypeptide can have an amino acid sequence with at least 45% sequence identity, e.g., 45%, 50%, 55%, 60%, 65%, 70%, 75%, 80%, 85%, 90%, 95%, 97%, 98%, or 99% sequence identity, to an amino acid sequence encoded by SEQ ID NOs: 1-8.

A virulence factor polypeptide encoded by a recombinant nucleic acid can be a native virulence factor polypeptide, i.e., one or more additional copies of the coding sequence for a virulence factor polypeptide that is naturally present in the cell. Alternatively, a virulence factor polypeptide can be heterologous to the cell. For example, a C. albicans can contain the coding sequence for a virulence factor polypeptide from another fungal species, e.g., Candida albicans, C. dubliniensis, C. krusei, C. tropicalis, C. parapsilosis, C. glabrata.

A virulence factor polypeptide can include additional amino acids that are not involved in virulence, and thus can be longer than would otherwise be the case. For example, a virulence factor polypeptide can include an amino acid sequence that functions as a reporter. Such a virulence factor polypeptide can be a fusion protein in which a green fluorescent protein (GFP), yellow fluorescent protein (YFP), or red fluorescent protein (YFP) polypeptide is fused to, e.g., SEQ ID NOs: 1-8.

Virulence factor polypeptide suitable for use in the invention can be identified by analysis of nucleotide and polypeptide sequence alignments. For example, performing a query on a database of nucleotide or polypeptide sequences can identify homologs and/or orthologs of virulence factor polypeptides. Sequence analysis can involve BLAST, Reciprocal BLAST, or PSI-BLAST analysis of nonredundant databases using known Virulence factor polypeptide amino acid sequences. Those polypeptides in the database that have greater than 40% sequence identity can be identified as candidates for further evaluation for suitability as a virulence factor polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains suspected of being present in virulence factor polypeptides, e.g., conserved functional domains.

The identification of conserved regions in a template or subject polypeptide can facilitate production of variants of wild type virulence factor polypeptides. Conserved regions can be identified by locating a region within the primary amino acid sequence of a template polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains at sanger.ac.uk/Pfam and genome.wustl.edu/Pfam. A description of the information included at the Pfam database is described in Sonnhammer et al., (Nucl. Acids Res., 26:320-322, 1998); Sonnhammer et al. (Proteins 28:405-420, 1997); and Bateman et al. (Nuel. Acids Res. 27:260-262, 1999).

Conserved regions also can be determined by aligning sequences of the same or related polypeptides from closely related species. Closely related species preferably are from the same family. In some embodiments, alignment of sequences from two different species is adequate. For example, sequences from C. albicans and C. krusei can be used to identify one or more conserved regions.

Typically, polypeptides that exhibit at least about 40% amino acid sequence identity are useful to identify conserved regions. Conserved regions of related polypeptides can exhibit at least 45% amino acid sequence identity (e.g., at least 50%, at least 60%, at least 70%, at least 80%, or at least 90% amino acid sequence identity). In some embodiments, a conserved region of target and template polypeptides exhibit at least 92%, 94%, 96%, 98%, or 99% amino acid sequence identity. Amino acid sequence identity can be deduced from amino acid or nucleotide sequences. In certain cases, highly conserved domains have been identified within virulence factor polypeptides. These conserved regions can be useful in identifying functionally similar orthologous virulence factor polypeptides.

In some instances, suitable virulence factor polypeptides can be synthesized on the basis of consensus functional domains and/or conserved regions in polypeptides that are homologous virulence factor polypeptides. Domains are groups of substantially contiguous amino acids in a polypeptide that can be used to characterize protein families and/or parts of proteins. Such domains have a “fingerprint” or “signature” that can comprise conserved (1) primary sequence, (2) secondary structure, and/or (3) three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Nucleic Acids:

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. These types of nucleic acids can be tested as potential antifungal agents.

An “isolated” nucleic acid can be, for example, a naturally-occurring DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Isolated nucleic acid molecules can be produced by standard techniques. For example, polymerase chain reaction (PCR) techniques can be used to obtain an isolated nucleic acid containing a nucleotide sequence described herein. PCR can be used to amplify specific sequences from DNA as well as RNA, including sequences from total genomic DNA or total cellular RNA. Various PCR methods are described, for example, in PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid. Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring DNA.

As used herein, the term “percent sequence identity” refers to the degree of identity between any given query sequence and a subject sequence. A subject sequence typically has a length that is more than 80 percent, e.g., more than 82, 85, 87, 89, 90, 93, 95, 97, 99, 100, 105, 110, 115, or 120 percent, of the length of the query sequence. A query nucleic acid or amino acid sequence is aligned to one or more subject nucleic acid or amino acid sequences using the computer program ClustalW (version 1.83, default parameters), which allows alignments of nucleic acid or protein sequences to be carried out across their entire length (global alignment). See Chema et al., Nucleic Acids Res. 31(13):3497-500, 2003.

The term “exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor and not into the cell under consideration.

Recombinant constructs can be used to transform fungal cells in order to modulate virulence factor levels. A recombinant nucleic acid construct can comprise a nucleic acid encoding a virulence factor polypeptide as described herein, operably linked to a regulatory region suitable for expressing the virulence factor polypeptide in a cell. Thus, a nucleic acid can comprise a coding sequence that encodes any of the virulence factor polypeptides as set forth in SEQ ID NOs: 1-8.

In some cases, a recombinant nucleic acid construct can include a nucleic acid comprising less than the full-length coding sequence of a virulence factor polypeptide. For example, a recombinant nucleic acid construct can comprise a virulence factor nucleic acid having the nucleotide sequence set forth in SEQ ID NO: 1-8. In some cases, a recombinant nucleic acid construct can include a nucleic acid comprising a coding sequence, a gene, or a fragment of a coding sequence or gene in an antisense orientation so that the antisense strand of RNA is transcribed.

It will be appreciated that a number of nucleic acids can encode a polypeptide having a particular amino acid sequence. The degeneracy of the genetic code is well known to the art. For example, codons in the coding sequence for a given virulence factor polypeptide can be modified such that optimal expression in a particular fungal species is obtained, using appropriate codon bias tables for that species.

Vectors:

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a fungal or host cell. For example, a marker can confer biocide resistance, such as resistance to an antibiotic (e.g., kanamycin, G418, bleomycin, or hygromycin), or an herbicide (e.g., chlorosulfuron or phosphinothricin). In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

Regulatory Regions:

The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence. Some suitable promoters initiate transcription only, or predominantly, in certain cell types.

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences.

It will be understood that more than one regulatory region may be present in a recombinant polynucleotide, e.g., introns, enhancers, upstream activation regions, transcription terminators, and inducible elements. Thus, more than one regulatory region can be operably linked to the sequence of a polynucleotide encoding a virulence factor polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation

Agents:

The model systems can also include an agent that inhibits the expression of a gene or the activity of a gene product in the C. elegans or the human pathogen. The agent can vary depending on the organism and the particular gene product. An agent can be a nucleic acid sequence, a polypeptide, or a small molecule. In some embodiments, the model system can include a combination of two or more different agents, for example, a nucleic acid and a small molecule, a nulceic acid and a polypeptide, a small molecule and a polypeptide.

A nucleic acid agent can be a nucleic acid expressed by the C. elegans, the human pathogen, or, in the event the food source is a heterologous cell, by the food source. A number of nucleic acid based methods, including antisense RNA, co-suppression, ribozyme directed RNA cleavage, and RNA interference (RNAi) can be used to inhibit protein expression in host cells, e.g., C. elegans and fungal pathogens, e.g., Candida spp. The methods descibed below can be used to inhibit, for example, expression of any of the C. albicans polypeptides encoded by SEQ ID NOs.: 1-8. Antisense technology is one well-known method. In this method, a nucleic acid segment from a gene to be repressed is cloned and operably linked to a promoter so that the antisense strand of RNA is transcribed. The recombinant vector is introduced into the appropriate cell, as described above, and the antisense strand of RNA is produced. The nucleic acid segment need not be the entire sequence of the gene to be repressed, but typically will be substantially complementary to at least a portion of the sense strand of the gene to be repressed. Generally, higher homology can be used to compensate for the use of a shorter sequence. Typically, a sequence of at least 30 nucleotides is used, e.g., at least 40, 50, 80, 100, 200, 500 nucleotides or more.

Constructs containing operably linked nucleic acid molecules in the sense orientation can also be used to inhibit the expression of a gene. The transcription product can be similar or identical to the sense coding sequence of a polypeptide of interest. The transcription product can also be unpolyadenylated, lack a 5′ cap structure, or contain an unsplicable intron.

In another method, a nucleic acid can be transcribed into a ribozyme, or catalytic RNA, that affects expression of an mRNA. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide.

Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contain a 5′-UG-3′ nucleotide sequence. The construction and production of hammerhead ribozymes is known in the art. Hammerhead ribozyme sequences can be embedded in a stable RNA such as a transfer RNA (tRNA) to increase cleavage efficiency in vivo.

RNAi can also be used to inhibit the expression of a gene. For example, a construct can be prepared that includes a sequence that is transcribed into an interfering RNA. Such an RNA can be one that can anneal to itself, e.g., a double stranded RNA having a stem-loop structure. One strand of the stem portion of a double stranded RNA comprises a sequence that is similar or identical to the sense coding sequence of the polypeptide of interest, and that is from about 10 nucleotides to about 2,500 nucleotides in length. The length of the sequence that is similar or identical to the sense coding sequence can be from 10 nucleotides to 500 nucleotides, from 15 nucleotides to 300 nucleotides, from 20 nucleotides to 100 nucleotides, or from 25 nucleotides to 100 nucleotides. The other strand of the stem portion of a double stranded RNA comprises a sequence that is similar or identical to the antisense strand of the coding sequence of the polypeptide of interest, and can have a length that is shorter, the same as, or longer than the corresponding length of the sense sequence. The loop portion of a double stranded RNA can be from 10 nucleotides to 5,000 nucleotides, e.g., from 15 nucleotides to 1,000 nucleotides, from 20 nucleotides to 500 nucleotides, or from 25 nucleotides to 200 nucleotides. The loop portion of the RNA can include an intron. A construct including a sequence that is transcribed into an interfering RNA is transformed into plants as described above. Methods for using RNAi to inhibit the expression of a gene are known to those of skill in the art.

In some nucleic-acid based methods for inhibition of gene expression, a suitable nucleic acid can be a nucleic acid analog. Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-deoxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered morpholino ring, or peptide nucleic acids, in which the deoxyphosphate backbone is replaced by a pseudopeptide backbone and the four bases are retained. See, for example, Summerton and Weller (Antisense Nucleic Acid Drug Dev. 7:187-195, 1997); and Hyrup et al., Bioorgan. Med. Chem. 4:5-23, 1996). In addition, the deoxyphosphate backbone can be replaced with, for example, a phosphorothioate or phosphorodithioate backbone, a phosphoroamidite, or an alkyl phosphotriester backbone.

Protein expression can also be modulated using specific agents, e.g., molecules that inhibit enzyme activity. Exemplary inhibitors of NADPH oxidase include apocynin and DPI (diphenylene iodonium).

Kits:

The compositions described herein can also be assembled in kits, together with packaging materials and instructions for use. For example, the kits can include measured amounts of the multi-organism model system of comprising the nematode, C. elegans, a fungal pathogen that infects humans, and a source of food for the C. elegans. The components, e.g., the nematode C. elegans, a fungal pathogen that infects humans, and/or a source of food for the C. elegans can be packed separately or together. The fungal pathogen can be any one or more of those disclosed herein. Both wild type and/or mutants organisms can be included. For example, the C. elegans can be a wild type C. elegans and the pathogen can comprise a mutant gene. Alternatively, or in addition, the C. elegans can be a mutant C. elegans and the pathogen can be a wild type pathogen. The kit can include or exclude one or more of the components.

The instructions for use can be conveyed by any suitable media. For example, they can be printed on a paper insert in one or more languages or supplied audibly or visually (e.g., on a compact disc). The packaging materials can include packaging materials, for example, vials, packets, containers. The components of the kit may be suitable for immediate use. The invention encompasses kits, however, that include concentrated or lyophilized organisms or formulations and/or materials that may require dilution prior to use.

C. albicans mucosal infections have not been studied genetically because of the lack of an appropriate model system. The widely accepted mouse tail vein injection model mimics dissemination and organ pathogenesis of C. albicans but does not recapitulate initial infection because an overwhelming number of colony-forming units are introduced into the bloodstream, and this allows them to bypass the innate immune system of the host. Furthermore, these murine models are expensive and not suitable for large-scale mutant screens.

A variety of in vitro, ex vivo, and in vivo models have been employed to study the interaction between the host and this fungal pathogen. Fully in vitro studies of hyphal morphogenesis and biofilm formation, amongst many others, have yielded important insights into virulence (Finkel et al., Nat. Rev. Microbiol. 9:109-118, 2011; Nobile et. al., Curr. Biol. 15:1150-1155, 2005). Ex vivo models, co-culturing C. albicans with isolated macrophages, neutrophils, epithelial or endothelial cells, and even intact, perfused organs, have demonstrated that C. albicans has very complex responses to host cell contact, which can differ dramatically between cell types (Fradin et al., Mol. Microbiol. 56:397-415, 2005; Lorenz et al., Eukaryot. Cell. 3:1076-1087, 2004; Park et al., Eukaryot. Cell. 8:1498-1510, 2009; Rubin-Bejerano et al., Proc. Natl. Acad. Sci. U.S.A. 100:11007-11012, 2003; Thewes et al., Mol. Microbiol. 63:1606-1628, 2007; Zakikhany et al., Cell. Microbiol. 9:2938-2954, 2007). A murine model of disseminated candidiasis has been frequently used to validate the role of specific genes on overall virulence. There is a general appreciation that each of these models has provided important insights into fungal pathogenesis. Recently, invertebrate models have become additional tools to dissect the roles of components of the antifungal host defense system, including flies, wax moth (Galleria melonella) larvae, and the nematode Caenorhabditis elegans (Brennan et al., FEMS Immunol. Med. Microbiol. 34:153-157, 2002; Chamilos et al., J. Infect. Dis. 193:1014-1022, 2006; Pukkila-Worley et al., Eukaryot. Cell. 8:1750-1758, 2009).

C. elegans has emerged as a useful model to study infectious disease for several reasons. First, facets of its innate immune system are conserved in humans (Kim et al., Science 297:623-626, 2002; Mallo et al., Curr. Biol. 12:1209-1214, 2002) and the nematode reacts to pathogens in a manner similar to mammals, such as activation of specific signal transduction pathways (Jain et al., Eukaryot. Cell. 8:1218-1227, 2009; Kim et al., Science 297:623-626, 2002; Mallo et al., Curr. Biol. 12:1209-1214, 2002; Pukkila-Worley et al., PLoS. Pathog. 7:e1002074). A rich body of literature demonstrates that human pathogens, both bacteria and fungi, also infect C. elegans in ways that are mechanistically similar to humans. For example, opportunistic human pathogens, Pseudomonas aeruginosa (Darby et al., Proc. Natl. Acad. Sci. U.S.A. 96:15202-15207, 1999; Kim et al., Science 297:623-626, 2002; Mahajan-Miklos et al., Cell. 96:47-56, 1999; Tan et al., Proc. Natl. Acad. Sci. USA 96:715-720, 1999; Tan et al., Proc. Natl. Acad. Sci. USA 96:2408-2413, 1999) and Serratia marcescens (Kurz et al., EMBO J. 22:1451-1460, 2003; Mallo et al., Curr. Biol. 12:1209-1214, 2002) both produce toxins that are required for pathogenesis in disparate eukaryotic hosts. Mutant studies in Salmonella typhimurium, typically thought to have a narrow host range, shows a direct correlation in virulence between humans and C. elegans (Aballay et al., Curr. Biol. 10:1539-1542, 2000; Labrousse et al., Curr. Biol. 10:1543-1545, 2000). More recently a comparative study in C. elegans using fungal pathogens of the genus Cryptococcus (Mylonakis et al., Proc. Natl. Acad. Sci. USA 99:15675-15680, 2002; Mylonakis et al., Mol. Microbiol. 54:407-419, 2004) showed that only the human pathogen C. neoformans, but not other related yeasts (C. kurtzingii or C. laurentii) killed the nematode. Furthermore these studies demonstrated that a virulence factor such as Kin1, first identified in nematodes was also important in mammals (Nakagawa et al. Microbiol. Immunol. 47:395-403, 2003). More recent whole genome analyses of C. elegans infected with C. albicans reveal that the nematode induces immune defenses with known antifungal properties (Pukkila-Worley et al., PLoS. Pathog. 7:e1002074).

C. elegans infected with bacterial pathogens reveal that generation of reactive oxygen species is an important part of the nematode's defense response (Chavez et al., Infect. Immun. 77:4983-4989, 2009; Jain et al., Eukaryot. Cell. 8:1218-1227, 2009), a hallmark shared with mammalian innate immune responses. We previously reported a C. elegans-based assay to study several aspects of disease progression, namely: deformity in the anal region (Dar), an early marker of infection; intestinal distension, resulting from colonization of the intestine; swelling in the vulva, representing infection of other epithelial layers, and ultimately death of the host worm (Jain et al., Eukaryot. Cell. 8:1218-1227, 2009). Developed initially using Saccharomyces cerevisiae, here we have adapted the assay for C. albicans and, using genetic tools available in both the fungus and nematode, are able to dissect the role of ROS in antifungal innate immunity. We employ genetic and pharmacological tools to alter the intricate balance between the host and the pathogen and demonstrate that ablating either the ability of the worm to produce ROS or C. albicans to detoxify it (via mutation of the Cap1 transcription factor) has dramatic effects on the outcome of this infection. Results in the worm were recapitulated in a macrophage co-cultures, validating this model. Surprisingly, however, the C. albicans cap1Δ mutant retained virulence in the disseminated murine bloodstream model, suggesting that additional layers of regulation of antioxidant defense exist in the context of a mammal. This work thus provides an avenue to investigate fungal pathogenesis and has allowed us to identify further complexity in the C. albicans-host interaction.

Mammalian biology has been effectively modeled using a variety of species that present substantial advantages in complexity, genetic tractability, ethical considerations and cost. The last decade has seen the acceptance of invertebrates such as C. elegans as relevant hosts that can contribute to molecular understanding of microbial pathogenesis. The C. elegans model, which has been generally used to study systemic pathogens such as Cryptococcus, Pseudomonas and Enterococcus, clearly differs from mammalian infection in several respects, including the site of infection (gut versus bloodstream), growth temperature (25-30° C. vs. 37° C.) and the absence of an adaptive immune response. Yet, equally clearly, C. elegans and other invertebrates have provided important insights into bacterial and fungal pathogenesis and many mutants have been uncovered that attenuate virulence both in the worm gut and in the mouse bloodstream (Kurz et al., Trends Microbiol. 8:142-144, 2000; Maccallum, Int. J. Microbiol. 2012:363764, 2012; Pradel et al., Annu. Rev. Genet. 38:347-363, 2004). We show here a case in which there is a difference between mammalian and nematode models with the C. albicans cap1Δ/Δ strain, yet even this is instructive and the worm offers opportunities to dissect the important components of the innate immune response to fungal pathogens.

We have previously demonstrated that the model yeast S. cerevisiae can cause pathology in the nematode (Jain et al., Eukaryot. Cell. 8:1218-1227, 2009). In this work we show that this assay is far more robust when using pathogenic Candida species and that the virulence of these species in the worm roughly correlates with virulence in mice. The worm has emerged as a valuable model to investigate host responses because aspects of its innate immune system are conserved in humans and our nematode infection assay faithfully recapitulated the ROS-mediated innate response observed in humans.

Generation of ROS has been shown to be an important component of the worm's defense against bacterial infections (Chavez et al., Infect. Immun. 77:4983-4989, 2009) and we demonstrate that here for fungal infections as well. Data presented here indicates that Cap1, a fungal-specific transcription factor known to regulate oxidative stress responses (Alarco et al., J. Bacteriol. 181:700-708.3, 1999; Wang et al., Free Radic. Biol. Med. 40:1201-1209, 2006) and drug resistance pumps (Alarco et al., J. Biol. Chem. 272:19304-19313, 1997), is required for virulence of C. albicans in nematodes and cultured macrophages. Specifically, strains lacking CAP1 are induce the DAR phenotype less frequently and attenuate virulence in a worm killing assay relative to the wild-type strain. This is directly due to the increased sensitivity of this mutant to ROS because mutant worms that cannot produce ROS due to a mutation in the host oxidase do not show early signs of infection nor do they succumb to an infection with the cap1Δ/Δ null mutant. Furthermore when the NADPH oxidase is chemically inactivated in cultured macrophages, cap1Δ/Δ null mutants are able to survive just as well as wild-type Candida.

Given the data above and the known in vitro functions of CAP1, the results of the murine model are surprising, as it is difficult to conclude that loss of CAP1 alters virulence. Since some other antioxidant proteins, such as the secreted superoxide dismutase SOD5 and catalase CAT1, are attenuated in this same model (Martchenko et al., Mol. Biol. Cell. 15:456-467, 2004; Nakagawa et al., Microbiol. Immunol. 47:395-403, 2003; Wysong et al., Infect. Immun. 66:1953-1961, 1998) it seems clear that anti-oxidant defenses are important in vivo. There is a small difference in the mutant and wild-type strain, but the difference is not eliminated in the complemented strain. It is possible that the reintroduction of a single copy of CAP1 is haploinsufficient, though this strain fully complements all in vitro phenotypes. A more intriguing possibility is that there may be Cap1-independent mechanisms for their induction in the context of the mammalian host. Indeed, SOD4 and SOD5 are both more highly expressed in hyphal cells, which can be induced in response to several host-associated cues (Frohner et al., Mol. Microbiol. 71:240-252, 2009; Martchenko et al., Mol. Biol. Cell. 15:456-467, 2004). Thus, our C. elegans model may allow analysis of critical fungal responses to the innate immune system that could be masked by phenotypic redundancy in the context of the intact mammal. Genetic tractability is a key advantage of the C. elegans assay, even over other invertebrates, and our model is intended to maximize the potential of the nematode. By feeding C. elegans on C. albicans, we avoid manipulation of individual animals, a time-consuming aspect of screens in both vertebrate and other invertebrate species such as mice, zebrafish, Drosophila or Galleria larvae. Further, our virulence assay can be differentiated from others C. elegans-based assays because we include E. coli as the primary nutritional source and spike in C. albicans as the infectious agent. This avoids any complicating factors that may come from different nutritional states, as we have found that C. elegans does not feed well on a lawn of fungal cells alone. In addition, this system can be easily adapted to conduct systematic mutant screens for modulators of the innate immune response using the existing RNAi knockdown collection, available as E. coli strains expressing double stranded RNA that is specific to any C. elegans gene, which knock down the gene's protein concentration to a negligible amount. In conclusion, we have shown that this model system offers a robust means to probe both innate immunity and fungal pathogenesis.

### EXAMPLES

### Example 1

### Materials And Methods

Strains, Media and Growth Conditions:

The C. albicans strains used are listed in Table 1 and are based on SC5314 and its auxotrophic derivatives CAI4-F2. C. albicans transformations were performed via electroporation (Reuss et al., Gene 341:119-127 (2004)). Fungal growth medium was prepared as described previously (Sherman et al., (1986)) and strains were grown overnight in yeast extract-peptone-dextrose (YPD) at 37° C. The C. elegans strains were grown at 20° C. on nematode growth agar medium (NGM) spotted with Escherichia coli OP50 and maintained as described previously (Brenner, Genetics 77:71-94 (1974)). E. coli OP50 was grown overnight in Luria broth at 37° C.

TABLE 1
Strains used in this study.

| | | | |
|---|---|---|---|
| | Relevant | | |
| Strains | Genotype | Complete Genotype | Source |

C. elegans strains

| | | | |
|---|---|---|---|
| N2 Bristol | Wildtype | Wild type | Brenner, Genetics |
| | | | 77: 71-94 (1974) |
| CB767 | bli-3 | bli-3(e767)I | Brenner, Genetics |
| | | | 77: 71-94 (1974) |

S. cerevisiae strains

| | | | |
|---|---|---|---|
| BY4741 | Wildtype | MATa; his3Δ1; leu2Δ0; met15Δ0; ura3Δ0 | Brachmann, C. B. |
| | | | et al., Yeast. 14: |
| | | | 115-132 (1998) |

C. albicans strains

| | | | |
|---|---|---|---|
| SC 5314 | Wild type | Wild type | Fonzi, C. et al., |
| | | | Genetics. 143: 717-728 |
| | | | (1993) |
| Caf 2-1 | URA3/ura3Δ | URA3/ura3Δ::imm434 | Fonzi, C. et al., |
| | | | Genetics. 143: 717-728 |
| | | | (1993) |
| CJD21 | cap1Δ/Δ | cap1Δ::hisG/cap1Δ::hisG-URA3-hisG | Alarco et al., J. |
| | | ura3Δ/ura3Δ | Bacteriol. 181: |
| | | | 700-708.3 (1999) |
| AGC2 | cap1Δ/Δ | cap1Δ::hisG/cap1Δ::hisG ura3Δ/ura3Δ | This study |
| | | RPS10/rps1::URA3-CIP10 | |
| AGC4 | cap1Δ/Δ | cap1Δ::hisG/cap1Δ::hisG ura3Δ/ura3Δ | This study |
| | complement | RPS10/rps1::CAP1-URA3-CIP10 | |
| | efg1Δ/Δ | efg1Δ::hisG/efg1Δ::hisG-URA3-hisG | Lo et al., Cell |
| | | ura3Δ/ura3Δ | 90: 939-949 (1997) |
| | cph1Δ/Δ | cph1Δ::hisG/cph1Δ::hisG-URA3-hisG | Lo et al., Cell |
| | | ura3Δ/ura3Δ | 90: 939-949 (1997) |
| | efg1Δ/Δ | efg1Δ::hisG/efg1Δ::hisG-URA3-hisG | Lo et al., Cell |
| | cph1Δ/Δ | cph1Δ::hisG/cph1Δ::hisG ura3Δ/ura3Δ | 90: 939-949 (1997) |

Other Candida strains

| | | | |
|---|---|---|---|
| C. albicans | Wild type | Clinical isolates | White, T. C., |
| | | | Atimicrob Agents |
| | | | Chemother. |
| | | | 41: 1482-1487 |
| | | | (1997) |
| C. dubliniensis | Wild type | Clinical isolates | Moran, G. P. et al., |
| | | | Antimicrob Agents |
| | | | Chemother. |
| | | | 41: 617-623 (1997) |
| C. krusei | Wild type | Clinical isolates | A.B. |
| | | | Onderdonk |
| C. tropicalis | Wild type | Clinical isolates | A.B. |
| | | | Onderdonk |
| C. parapsilosis | Wild type | Clinical isolates | Kuhn, D. M. et al., |
| | | | Emerg Infect Dis. |
| | | | 10: 1074-1081 |
| | | | (2004); and |
| | | | Laffey, S. F. et al., |
| | | | Microbiology. |
| | | | 151: 1073-1081 |
| | | | (2005) |
| C. glabrata | Wild type | Clinical isolates | Mundy, R. D. et al., |
| | | | J Infect Dis. |
| | | | 199: 1891-1898 |
| | | | (2009) |

Generation of cap1 Mutant Strains:

The existing cap1Δ/Δ strain CJD21 (Alarco et al., J. Bacteriol. 181:700-708.3, 1999) expresses URA3 from the disrupted cap1 locus, a strategy that has been subsequently demonstrated to potentially affect virulence through misexpression of the URA3 marker (Lay et al., Infect. Immun. 66:5301-5306, 1998; Sundstrom et al., Infect. Immun. 70:3281-3283, 2002). This can be overcome through ectopic integration of URA3 at the RPS10 locus using plasmid CIp10 (Brand et al., Eukaryot. Cell. 3:900-909, 2004; Murad et al., Yeast 16:325-327, 2000). To generate cap1Δ/Δ mutant and complemented strains with URA3 at RPS 10, we grew CJD21 on YPD for two overnight passages in YPD, then plated to media containing 5-fluororotic acid (5-FOA) to select for ura3 auxotrophs (Boeke et al., Methods Enzymol. 154:164-175, 1987). PCR amplified the CAP1 open reading frame from genomic DNA of strain SC5314, plus ˜1,000 bp of 5′ UTR and ˜350 bp of 3′UTR. This fragment was ligated between the XhoI and HinDIII sites in CIp10. This plasmid was digested with StuI and used to transform the 5-FOA-selected cap1Δ/Δ strain. In parallel, the empty CIp10 plasmid was digested with StuI and used to transform the same strain. After selection on SD-Ura, correct integration at RPS10 was confirmed by PCR to generate cap1Δ/Δ strains with either URA3 or CAP1-URA3 expressed from the same genomic site as the mutant (AGC2) and complemented strains (AGC4), respectively.

Microscopic Analysis of C. elegans:

A 2% agarose pad containing 0.01 M sodium azide as anesthetic was prepared on a slide. A 5 μl drop of M9 buffer was added to the pad. Worms exposed to wild type Candida or RFP-labeled Candida were picked and transferred to the drop on the slide. Mounted worms were then covered with a coverslip and observed at 200× and 400× magnifications using an Axiovision Zeiss microscope under differential interference contrast (DIC; Nomarski) and epifluorescence optics. An ApoTome attachment was used to enhance the fluorescence images.

Egg Preparation:

Three worms in the L3/L4 stage each were transferred to two NGM agar plates containing E. coli OP50 and grown at 20° C. for four days. On the day of the experiment, worms were washed off the plates with M9 buffer and centrifuged at 900×g for 2 minutes. The supernatant was removed, and the worms were then resuspended in a bleach solution (1:4 dilution of commercial bleach (5.25%) containing 0.25 M sodium hydroxide). The worm suspension was mixed gently by inversion for 3 minutes, and centrifuged for 2 minutes at 2,000×g. The pellet was washed and centrifuged with M9 buffer at 2,000×g for 2 minutes and then finally resuspended in 500 μl M9 buffer. The egg suspension was diluted or concentrated with M9 buffer as required to obtain approximately 30-40 eggs/5 μl.

Pathogenesis Assay:

E. coli and Candida strains were grown overnight at 37° C. Culture aliquots were centrifuged at full speed for 1 minute in a table top microcentrifuge, and the supernatant was removed. Pellets were washed twice in sterile deionized water, and finally resuspended to a final concentration of 200 mg/ml and 10 mg/ml, respectively. A mixture of 10 μl of a 50-mg/ml streptomycin sulfate stock to inhibit E. coli growth, 7 μl of distilled water, 2.5 μl of E. coli and 0.5 μl of Candida was spotted on to each NGM plate. E. coli spotted plates were used as control. Finally, 5 μl of C. elegans egg suspension was transferred to each plate. Plates were then kept in a 20° C. incubator and were observed over the next 5 days. All the experiments were done in triplicate. Student t-test was used to check the statistical significance of the differences observed between wild type and other Candida strains.

Macrophage Growth Inhibition Assay:

Macrophage cell line RAW 264.7 (ATCC) was used in the assay. The cell line was maintained in DMEM supplemented with 10% fetal bovine serum (FBS). The protocol was slightly modified from the original (Lopes da Rosa et al., Proc. Natl. Acad. Sci. U.S.A. 107:1594-1599, 2010). Briefly, macrophages on reaching 80-90% confluence were scraped and brought up in DMEM supplemented with 10% FBS and 100 U/ml penicillin and 100 μg/ml streptomycin. 2×10⁶ macrophage cells were plated in a 35 mm² plate and allowed to adhere for 5 hours. Candida strains were grown overnight at 37° C., diluted 1:10, and allowed to grow for another 5 hours. Candida cells were then washed with supplemented DMEM and were added to the plates containing macrophages in a ratio of 1:15 macrophage and to a final volume of 2 ml. Yeast strain was also grown in parallel without macrophages to calculate % survival. The plates were incubated overnight at 37° C. and 5% CO₂. Cells were then brought up to 24 mls in a tube using 0.05% Triton X-100 (v/v) in water to osmotically lyse the macrophage cells. Dilutions were prepared and plated on a YPD plate and grown overnight at 37° C. Colony forming units (CFU) were counted and percentage survival was calculated by taking the ratio of CFU from co culture of Candida and macrophage to the CFU obtained for Candida alone.

Experiments involving diphenyleneiodium chloride (DPI) were performed as mentioned above but with the addition of 0.05 μM DPI from a 31.8 mM stock solution in DMSO. Controls without DPI had the same concentration of DMSO. Statistical analysis was done using Student's t-test.

Mouse Virulence Assay:

Mouse virulence assays were carried out as described previously (Ramirez et al., Eukaryot. Cell. 6:280-290, 2007). Female, adult (21-25 g) ICR mice (Harlan) were maintained on a normal lab diet. C. albicans strains were passaged twice in overnight cultures in YPD then diluted into fresh YPD and grown for three hours at 30° C. Cells were collected by centrifugation, washed with water, resuspended in PBS, then diluted and counted with a haemocytometer. Cells were diluted to 1×10⁸ cells/ml in PBS. Mice were injected with 100 μl of this suspension via the tail vein, with groups of 10 mice/strain. Animals were monitored 2-3 times daily for signs of infection and were euthanized when moribund. Survival data were analyzed with Prism5 (Graphpad Software) using the log rank test. All animal experiments were conducted in accordance with protocols approved by the University of Texas Health Science Center Animal Welfare Committee.

### Example 2

### Generation of Cap1 Homozygous and Complemented Strains

The original cap1Δ/Δ mutant strain CJD21 was generated by Raymond and colleagues (Alarco et al., J. Bacteriol. 181:700-708.3, 1999) using an approach that was subsequently shown to be inappropriate for animal experiments due to variability of expression of the URA3 selectable marker. We selected a ura3-derivative of the cap1Δ/Δ strain CJD21 on 5-FOA, and then integrated either URA3 or URA3-CAP1 at the RPS10 locus, using plasmid CIp10, a strategy shown to stably express URA3 during infection (Murad et al., Yeast 16:325-327, 2000). This generated a cap1Δ/Δ mutant strain (AGC2) and a complemented strain (AGC4); the mutant strain is phenotypically identical to the original CJD21 during in vitro challenge with ROS

### Example 3

### In Vivo Virulence Assay for Candida Infection

We have previously described a pathogenesis assay in which a small quantity of S. cerevisiae, a model pathogen was introduced with the nematode's standard diet of E. coli OP50. The E. coli was attenuated to avoid interactions with the fungal species under investigation (Jain et al., Eukaryot. Cell. 8:1218-1227, 2009). We uncovered molecular mechanisms of fungal virulence and the reciprocal innate immune response of nematodes, which is also conserved in mammals (Gauss et al., J. Leukoc. Biol. 82:729-741, 2007; Zu et al., J. Immunol. 160:1982-1989, 1998). Here we have adapted this assay to test various fungal pathogens of the Candida genus including C. albicans, C. dubliniensis, C. krusei, C. tropicalis, and C. glabrata. We decreased the concentration of Candida in the feeding mixture by 30-fold because of the increased pathogenicity of Candida species. Even at this lower fungal burden, several species induced Dar in 100% of the worms as compared to S. cerevisiae, which induced 0% Dar at the same concentration of inoculum (Table 2).

TABLE 2
Percentage DAR observed for different clinical isolates

| Strains | % Dar | n = |
|---|---|---|
| Candida | | |
| C. albicans (SC 5314) | 100 | 114 |
| C. albicans | 100 | 111 |
| C. dubliniensis | 100 | 122 |
| C. glabrata | 59.7 ± 1.1 (± SE) | 119 |
| C. parapsilosis | 4.9 ± 0.3 (± SE) | 133 |
| C. krusei | 100 | 115 |
| C. tropicalis | 100 | 134 |
| Saccharomyces | | |
| S. cerevisiae (BY4741) | 0 | 76 |

### Example 4

### Disease Phenotypes of C. elegans Upon C. albicans Infection

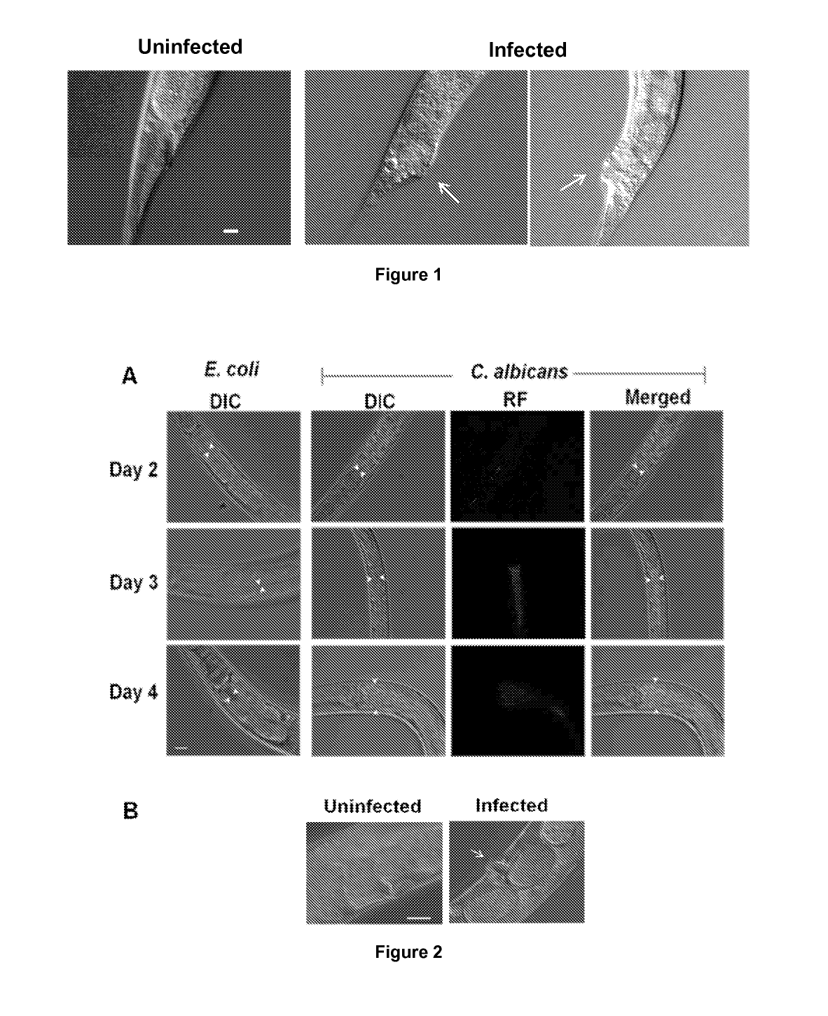

Subsequent studies were focused on C. albicans because it is the most prevalent infectious species in this genus (Gudlaugsson et al., Clin. Infect. Dis. 37:1172-1177, 2003) and adequate genomic and molecular tools have been developed. To visualize infection, disease progression, and death, we exposed nematodes to C. albicans and observed them daily over a 6-day period. The Dar phenotype was clearly visible in every worm on day 4 (FIG. 1 and Table 2). Swelling in the vulva region was also noted in the worms infected with C. albicans (FIG. 2B). The worm succumbed to the infection following these phenotypic observations. To observe colonization of the intestinal lumen, worms were exposed to mCherry labeled C. albicans (mCherry labeled SC5314, gift from Dr. Robert Wheeler, Univ. of Maine). Time-series micrographs of worms infected with C. albicans indicated that the intestinal lumen was colonized 2 days post exposure as compared to uninfected worms. Considerable intestinal distension was observed in infected worms on subsequent days (FIG. 2A) as compared to the uninfected control population. In general, the disease phenotypes were more robust with C. albicans as compared to S. cerevisiae, even with lower inoculum for infection. To further validate our assay we tested known mutants, efg1Δ and cph1Δ, previously documented virulence factors that regulate hyphal transition of C. albicans (Lo et al., Cell 90:939-949, 1997) and have been shown to be important in in vivo infections of mice and nematodes (Koh et al., PLoS. Pathog. 4:e35, 2008; Pukkila-Worley et al., PLoS. Pathog. 7:e1002074). The efg1Δ and cph1Δ single mutants showed decreased Dar, ˜10% and ˜50%, respectively, compared to the cognate wild type while efg1Δ cph1Δ double mutant failed to induce any Dar (0% Dar). These results recapitulate the pattern of virulence in mice, where the cph1Δ mutant is slightly attenuated, the efg1Δ mutant is significantly attenuated and the double mutant is completely avirulent (Lo et al., Cell 90:939-949, 1997). Accordingly, our assay is suitable for exploring virulence strategies of C. albicans and the reciprocal host defenses that maybe correlated to aspects of the innate immunity that conserved in mammals (Kim et al., Science 297:623-626, 2002; Mallo et al., Curr. Biol. 12:1209-1214, 2002).

### Example 5

### Cap1 is Required to Establish Infection in Nematodes

In vitro, Cap1 regulates the response to oxidative stress, which is a key component of the innate immune response to fungi (Alarco et al., J. Bacteriol. 181:700-708.3, 1999); Enjalbert et al., Mol. Biol. Cell. 17:1018-1032, 2006; Wang et al., Free Radic. Biol. Med. 40:1201-1209, 2006; Zhang et al., Mol. Microbiol. 36:618-629, 2000) and is induced in the presence of neutrophils (Fradin et al., Mol. Microbiol. 56:397-415, 2005). To correlate these studies performed at 30° C. and 37° C., respectively, with our in vivo infection performed at 20° C., we compared the ROS sensitivity profiles of cap1Δ/Δ, CAP1-complemented and the cognate wild type strain at 20° C., 30° C. and 37° C. Our reconstructed cap1Δ mutant failed to grow in the presence of hydrogen peroxide (at a final concentration of 2 mM) while the CAP1-complemented strain showed same growth as the wild type at all temperatures tested. These results indicate that the sensitivity to ROS of the C. albicans strains used in this study is independent of temperature.

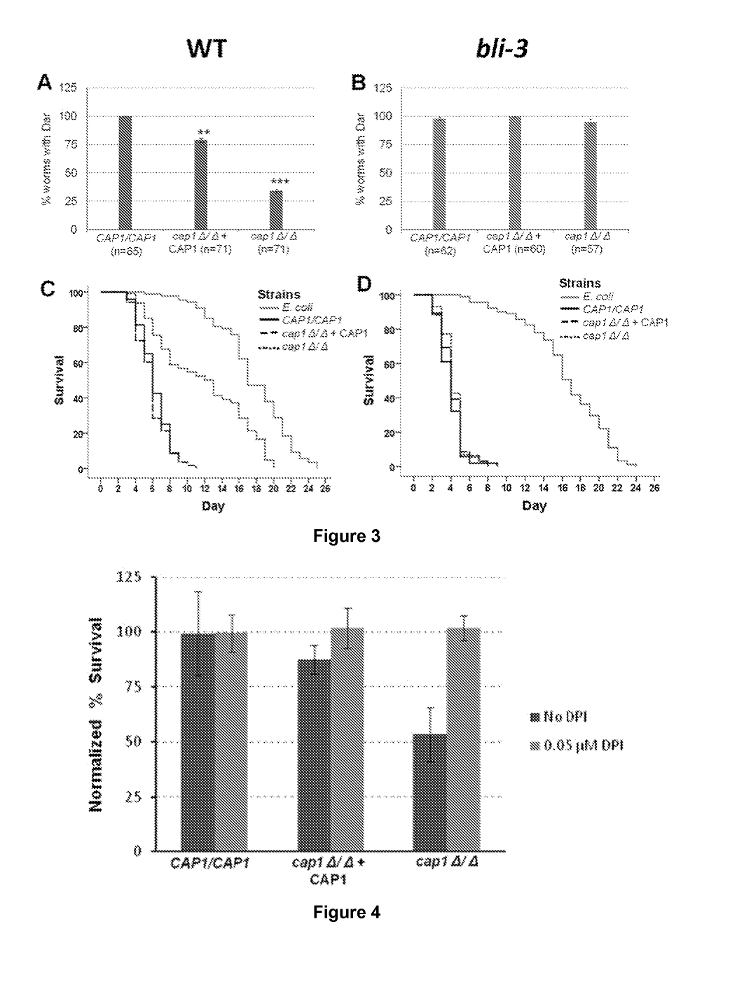

To test whether the mechanism by which CAP1 promotes virulence is related to the production of ROS by the host, we compared the Dar response of worms exposed to null mutant (cap1Δ/Δ) and wild type strain (CAP1/CAP1) as well as a CAP1-complemented strain (cap1Δ/Δ+CAP1) where a single wild type copy CAP1 is replaced at the native locus. The wild type strain was able to induce Dar in 100% of the worms while cap1 homozygous mutant showed a significant reduction in Dar induction (FIG. 3A). The CAP1-complemented strain showed an intermediate Dar response, commonly observed for complemented strains because a diploid deletion is complemented with a single copy. This result indicates that Cap1 function is required for full virulence in the nematode model.

The bli-3 gene contains the only NADPH oxidase moiety in C. elegans genome where it is found fused the peroxidase domain (hence called Dual oxidase, Duox) (Edens et al., J. Cell. Biol. 154:879-891, 2001). We and others have previously reported that Bli-3 produces ROS in response to pathogenic insult (Chavez et al., Infect. Immun. 77:4983-4989, 2009; Jain et al., Eukaryot. Cell. 8:1218-1227, 2009). To test the hypothesis that Cap1 is responsible for counteracting this oxidative environment upon C. albicans infection, we exposed bli-3 mutant worms to cap1Δ/Δ, CAP1-complemented and wild type CAP1/CAP1 strains. As shown in FIG. 3B, the cap1Δ/Δ null mutant is able to induce the Dar phenotype in bli-3 mutant worms to a degree indistinguishable from their wild type and complemented counterparts. Thus, Cap1 function is not required to infect bli-3 mutant worms, presumably because the generation of ROS, which is part of the host's defense repertoire, has been compromised in the mutant.

### Example 6

### Cap1 is Required to Sustain Infection and Ultimately Kill the Nematode Host

We and others have previously shown that the Dar phenotype is an early indicator of an eventually lethal infection, and Dar is reduced in worms exposed to the cap1Δ/Δ null mutant (Hodgkin et al., Curr. Biol. 10:1615-1618, 2000; Jain et al., Eukaryot. Cell. 8:1218-1227, 2009). To test the hypothesis that these mutants are avirulent, we measured the life span of worms infected with the cap1 null mutant, complemented and wild type strains relative to uninfected worms. The lifespan of C. elegans infected wild type C. albicans (CAP1/CAP1) or a CAP1-complemented strain was significantly shorter (50% worms were dead by day 6) than those infected with the cap1Δ/Δ null mutant (50% worms were dead by dayl3) (FIG. 3C). These data indicate Cap1 is required for the infection to persist and ultimately kill the hosting worms. We repeated this assay using the ROS-deficient bli-3 C. elegans mutant. These worms were slightly more sensitive to infection with C. albicans but, in the absence of ROS, there was no difference in the kinetics of killing between the cap1Δ/Δ mutant and the wild-type or complemented strains (FIG. 3D). The lifespan of uninfected bli-3 mutants is the same as their wild type counterparts (FIG. 3) eliminating confounding factors other than ROS production as a likely cause of death of infected worms.

### Example 7

### Cap1 is Responsible for Neutralizing ROS Produced by Phagocytes

Part of the mammalian host defense against Candida infection is production of ROS within phagosomes that have engulfed the fungal pathogen (Vazquez-Torres et al., Microbiol. Mol. Biol. Rev. 61:170-192, 1997). Wild type C. albicans can effectively neutralize these ROS to survive (Arana et al., Cell. Microbiol. 9:1647-1659 (2007); Frohner et al., Mol. Microbiol. 71:240-252 (2009); Lorenz et al., Eukaryot. Cell. 3:1076-1087 (2004); Wellington et al. Infect. Immun. 77:405-413 (2009); Wysong et al., Infect. Immun. 66:1953-1961 (1998) eventually cause the macrophages to lyse. To test whether Cap1 mediates this key response that allow C. albicans to withstand the oxidative environment, we co-cultured macrophages with either wild type or cap1Δ/Δ strains. Our data indicate that the colony forming units (CFU) of C. albicans recovered from macrophages were significantly lower for cap1Δ/Δ null mutant compared to the cognate wild type (FIG. 4); the complemented strain survived macrophage phagocytosis to an extent similar to the wild-type.

To further correlate the survival of phagocytized C. albicans, with ROS-production within the phagocytes, we chemically inhibited ROS-production in the phagosomes with Diphenyleneiodonium chloride (DPI) (Hancock et al., Biochem. J. 242:103-107 (1987). DPI specifically inhibits the NADPH oxidase (Babior, Curr. Opin. Immunol. 16:42-47 (2004), the homolog of Bli-3 responsible for ROS production in macrophages. In the presence of DPI, there was no difference in CFUs recovered from macrophages between the three strains, suggesting that disabling the host ROS-production machinery allows cap1Δ/Δ, which is otherwise attenuated, to survive within the phagosome. These results suggest that C. albicans relies on Cap1 to neutralize the oxidative environment within the phagosome and eliminating the source of ROS in turn eliminates the need for Cap1.

### Example 8

### CAP1 has a Limited Effect During Mammalian Infection

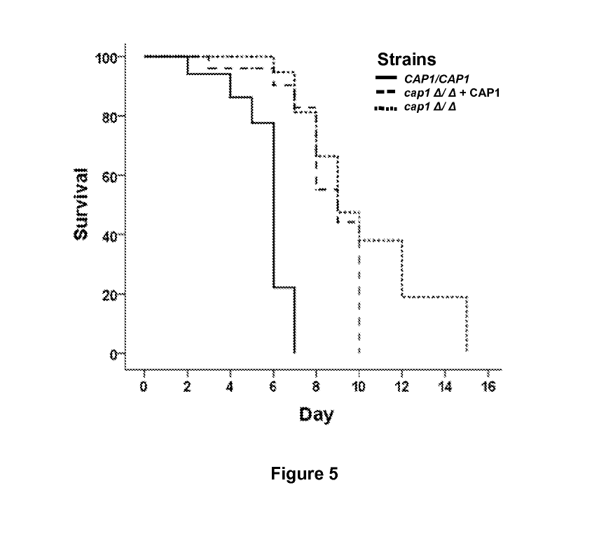

The cap1Δ/Δ mutant is clearly attenuated in the nematode model and during contact with macrophage and has previously been reported to be hypersensitive to several sources of oxidative stress (Alarco et al., J. Bacteriol. 181:700-708.3 (1999). We tested it in the standard mouse model of disseminated candidiasis, in which C. albicans is introduced directly into the circulatory system via the tail vein. We injected outbred ICR mice with 10⁶ C. albicans cells and monitored for signs of infection (FIG. 5; see Materials and Methods). Mice inoculated with the cap1Δ/Δ strain had a statistically significant increase in median survival times relative to wild-type (from 6.0 to 8.5 days, p=0.0003). However, the CAP1-complemented strain showed roughly the same virulence (median survival 8.0 days). Thus, despite the worm and macrophage data, in vitro cap1Δ/Δ phenotypes, and the general assumption that the oxidative burst is a key part of the anti-fungal innate immune response, we show that loss of CAP1 alters virulence only slightly.

### Example 9

### Virulence Screen

To test our virulence assay with true fungal pathogens, we used several clinical isolates of Candida. The results (Table 3) suggest that our assay is more robust with a true pathogen. Interestingly, C. glabrata, which is evolutionarily closer to S. cerevisiae, behaved more like S. cerevisiae than C. albicans. For further study we focused our attention on C. albicans. To validate the system, we performed a series of analogous experiments outlined in Jain et al. (2009). Briefly, to visualize Candida present in the intestine and clearly differentiate them from yeast sticking to the outside of the worm, we used a fluorescent-tagged housekeeping protein to follow progression of disease and to specifically test the hypothesis that the intestinal distension is due to the accumulation of yeast. Distension of the intestinal lumen was visible in DIC (Nomarski) micrographs of infected worms. To enhance visualization of the progression of disease and specifically test the hypothesis that the intestinal distension is due to the accumulation of Candida, we used a strain tagged with the Red Fluorescent Protein (RFP), obtained from Dr. R. Wheeler (Univ. of Maine, Orono). A time course of microscopic evaluation of infection using RFP-labeled candida revealed that progressively more RFP-labeled candida cells had accumulated in the pharynx and intestine, causing the lumen to be severely distended compared to uninfected worms, in which the intestinal lumen is a narrow tube. Furthermore a pronounced swelling in the vulval region was noted in worms.

TABLE 3
Percentage DAR observed for different clinical
isolates compared to S. cerevisiae and C. albicans lab strains.

| Strains | % Dar | n = |
|---|---|---|
| S. cerevisiae (BY4101) | 63 ± 1.5 | 80 |
| C. albicans (SC 5314) | 100 | 114 |
| C. albicans | 100 | 111 |
| C. dubliniensis | 100 | 122 |
| C. krusei | 100 | 115 |
| C. tropicalis | 100 | 134 |
| C. parapsilosis | 4.9 ± 0.3 | 133 |
| C. glabrata (BG1) | 59.7 ± 1.1 | 119 |

This assay was used to screen a library of C. albicans mutants (obtained from the fungal genetics stock center). Through such a screen, we are able to identify novel gene mutations in the C. albicans genome that hinder or prevent its ability to establish infection in a host organism (Table 4). We are currently verifying these findings in a null mutant. Furthermore, we are conducting phenotypic characterization of these mutants to focus our studies on the most promising candidates and/or the least understood ones.

The present invention encompasses the gene sequences identified in Table 2, vectors encoding them, engineered host cells containing them, the proteins encoded and methods of using the genes, expression vectors, host cells, and encoded proteins in assays of fungal pathogenesis and assays configured to identify putative anti-fungal agents. A pathway or protein that is absent in mammalian cells would be a potential drug target for antifungal therapeutics in multiple indications.

TABLE 4
Brief description of the putative (novel) virulence factors identified
in the mutant screen in C. albicans. Homologs were identified
in S. cerevisiae, and their descriptions are also noted.

| % Disease | C. albicans gene | S. cerevisiae homolog |
|---|---|---|
| 48 | DOT4: Cell surface | DOT4: Ubiquitin specific protease |
| | protein in hyphal | involved in sub-telomeric gene silencing |
| | cells similar to | |
| | Ubiquitine | |
| | hydrolase | |
| 30 | orf19.6713: | DEF1 (unclear): RNA PolII degradation |
| | Uncharacterized | factor |
| 36 | orf19.1219: | RKR1: RING domain ubiquitin ligase |
| | Uncharacterized | involved in chromatin modification |
| 47 | ZCF15: Predicted | PDR1 (ortholog): Transciption factor |
| | Zinc finger | important for drug resistance |
| | protein | |
| 68 | SAP8: Secreted | YPS1: GPI anchored aspartic protease |
| | Aspartyl Proteinase | important for cell wall growth and |
| | highly expressed | maintenance |
| | in opaque cells | |
| 44 | CMP1: Catalytic | CMP2: Catalytic subunit of calcineurin |
| | subunit of | |
| | calcineurin | |
| 46 | IFF11: Secreted | WSC4: ER membrane protein involved |
| | protein required | in translocation of secretory proteins |
| | for cell wall | |
| | structure | |

### Example 10

### C. albicans Genes that Relate to Fungal Virulence

The coding sequences of the genes identified in the screen described in Example 9 are listed below. The information was obtained from the Candida Genome Database on the world wide web (candidagenome.org). The identification numbers are those found on the database. One of skill in the art can access these genes and any homologues to design agents that can be used to modulate the expression of these genes.

| DOT4, coding sequence (exons only) DOT4/orf19.3370 | |
|---|---|
| (SEQ ID NO: 1) | |
| COORDS: Ca21chr4_C_albicans_SC5314: 721235-719028C Exon(s) | |
| only sequence (2208 nucleotides) | |
| orf19.3370\|DOT4 Length: 2208 Wed Mar. 7 08:29:37 2012 Type: N | |
| Check: 4670 . . . | |
| 1 | ATGTATCAAT CTTCATCTTC TAAACCAAAG CTAAACATTA ATAATAATTC |
| 51 | CACAACAATC ACCACTAATC CAACTACTAC CCTCAATCCT TTACTTTTTG |
| 101 | CAACCCCATT AAATTTCAAG CCAGCATCTA CATCCACCAC ATTAAGCACT |
| 151 | GGGTTTGATA ATCATATACC TAAATCATAC ATTGTTTTAT CAAATAACAG |
| 201 | CAACAACTCA AGTATGAAAA CTTCCAAGAA ACTTGGTAAA TCAAATACTA |
| 251 | AAAAAACTAC TAATGATCCA CCAAAACCTA AATCTTTAAA TGAAGCAATT |
| 301 | GCTAATTATA CTGGCAATAA ATATTTGACT CATAAGGAAA GGAAAAGGAA |
| 351 | AAGAAAATTA CAAGAAATAA TAACTGAAGA TAATCAAGTC CCCAAAGAAG |
| 401 | ATCAACAAGA ACAAGGTGAA GATGATGAAG AAGAAGAAGA AACCGATGAG |
| 451 | GCAAAACATG TAACTAAAAA AGTGAAAAAA GGAATGTGGG ATTCTATTAA |
| 501 | ATCATTCTGG CTGAGTACAC CTACTTCCAC ACCAACACCA CCTACAGAAG |
| 551 | AAGAAGAAGA AGATTTAGGA GAAAATATAG AAGAGAAGCA AGAGGAAGAA |
| 601 | GAGGAAGAGG AAGAAGAAGA AGACCATGAT TCAAGTTCTC CTGGTGAAGT |
| 651 | ATTTACTGAG ACATCACCAT TTACTGGATT TAGTGAACCA GAAAAAAGTG |
| 701 | AAATTGACGA AGAAGATGAA GAAGATGATG CAGATTTTGT GGATGAAGAG |
| 751 | TCAGGTTCAC CTGAAAGTGG TGATGAAGAT GATGAAGATG TTGAATCAGA |
| 801 | TTCTGTGTCA CCACAAGCAT CCGATGATGA CGATGAAGAT GAAGATTTAG |
| 851 | AAAAGTTAAA ATATGATTTA AAAATTGATC AATTAGTCAA CGACAAGGAT |
| 901 | GTCAATGAAG ATCAACAAGA GGAGGAGGAA GAGGAAGAGG AAGAGGAGGA |
| 951 | AACGCAAGAA CGGACATCAA CTACTCCACC AACTTCACCA GAAGAAGACA |
| 1001 | CAGAAAAGCA GCAACCAGAG CCACTGGAAG TTTCATACTA CGATATTAAT |
| 1051 | GAATATGAAG ATGATCGTGG GTCTAATAAT TCTGCTAGAA TATACAAAAA |
| 1101 | TTGGAGAGAA CTCGCTAACA AGAAACCTCC AGTTGGATTA TTGAATCATG |
| 1151 | GTGTCACTTG TTACATGAAT TCGGCAATTC AATCAATTAT TCATATACCG |
| 1201 | GCAATGCAAC ATTATTTAAA TGATGTGAAT GATAATAAAT ATTCTCAACT |
| 1251 | TAAACCAAGA TCAGTGACCC ATGTTTTAGC TGAATTATCT AAAAGAATGT |
| 1301 | GGTTAGCATC AAATACTAAC AACAAGAAGA AGGGTAAAAG TAATGGTGGA |
| 1351 | ATCAAATATA TTAATCCGAA AACAACAATT CAAAGATTGG ATGAAATTAA |
| 1401 | TTGCATGATG AGTGAATGGC AACAAGAAGA TTCTCATGAA TATTACATGT |
| 1451 | CATTAATCTC TAGACTACAA GAAGATTCTA CACCAAAGGG GAAAAAATTG |
| 1501 | AATGAATCGA TTATTTATGA TATCTTTGGT GGATTATTGA ATCAACGTAT |
| 1551 | TACGTGTCAT AAATGTCATA ATGTTTCAAT CACCAAGCAA GAATTTTATG |
| 1601 | ATTTATCATT AGGGTTAAAT AAGAAGAAGA TCAAATCAAA TAATAACGGC |
| 1651 | GACAACATTA CTAATGATAG CAATAATGAT ACCACATCCA CTACTGCCAC |
| 1701 | TTCCACCTCT ACCTCCGACC CAATCTTCAA TAGTAATAAA TTTTCTGTTG |
| 1751 | AAAAATCTAT TAATGATTTT TTCAGTAATG AGGTAATCAA GAAACTGGAA |
| 1801 | AATGATAGTA AATCAGGTTA TTTCTGTGAG AAATGTAATC GATTCACTCA |
| 1851 | TGCCAATAAA ATATCTTCCA TTGAAATTGC TCCAGAAACT TTAACCATTC |
| 1901 | ATTTCAAACG TTTCAAATTC AACAACAATT CTTCATCCAA AGTCAAACAG |
| 1951 | CTGATAAATT ATAGTAAATA CCTTGATTTA AGCAAATATA TGGTACAAGG |
| 2001 | TACAGCTAAA TATCGTTTGA CATCAGTTAT TGTTCATGAA GGAAGATCAA |
| 2051 | TTTCTAGTGG ACATTATGTT GCTCATTGTT TACAGCCTGA TGGATATACT |
| 2101 | TGGTGTACTT ATGATGATGA ATACATTAAT AAAATCGAGG AAAGAGTGGC |
| 2151 | ATTAAATGAT CCTTCTGCTT ATGTGTTGGT ATATACTAAA TTGACCCCAA |
| 2201 | AAGTTTAA |
| orf19.6713 coding sequence (exons only) (Has domain(s) with | |
| predicted nucleic acid binding, zinc ion binding activity and | |
| intracellular localization) | |
| (SEQ ID NO: 2) | |
| COORDS: Contig19-10254: 5809-4604C Exon(s) only sequence | |
| (1206 nucleotides) | |
| orf19.6713 Length: 1206 Wed Mar. 7 08:32:14 2012 Type: N | |
| Check: 561 . . . | |
| 1 | ATGTCGAGTG ATA7ACCTGA ACAAGAAATC CTAGATCCAA AGGATTCAAA |
| 51 | AAACGCTTCA TTGAATCTGA CAACGAAAAA GTCTCAAGAT AGCAAGCTTA |
| 101 | TGTTACCACC TTTAGGTGTC AAGTCTGAGC AACAACAACC ACAGCAACAG |
| 151 | CAACAAACCC CACTTTATGG TCAACAGGGG TCCCCGCAAG TTCAAACTCA |
| 201 | ACCGCAGTTT CAAGCAGACT ATTCGTATGC CAATTACCAA CCTCAAGCTC |
| 251 | AATTTTACGA CCCATATCAA AGACAAGTAA TTATGCAACA AGCCAATTTT |
| 301 | GCTTCACCAT CGCAATATCC AATTCAGCAG CCATATTCAT TTGCCAATGG |
| 351 | TATGACCAAC ATGGCCCAAT TCCACCCACA GACGTTGACA TCGCATTCCG |
| 401 | ACTTTGATCA ACAACAAATG CAGCAATCAA CCCAGCCATC GCAGCAAGTA |
| 451 | CAAGTACAGG TGCAGATACA ACCACAATCA AATCTGAACC TGTTCCCTGC |
| 501 | TGAGCAGAAA CGTGGAAGAA GGTTTAGAAG GAGATTCAAT CAAATTGAGA |
| 551 | GAAAATATCC TTGTTCGTTT CCTGGATGTC AAAAGAGTTA TGGTTCTTTG |
| 601 | AATCATTTAA ACACCCATAT TGTAACCAAA AAGCATGGTC ACAGAAAATC |
| 651 | AAAAGCTGAT TTCCAACATT CTCTTCAATC AAAAGAAGAA AACAAACCAT |
| 701 | CAGAAATTTC ACAATACGTT CAGCCTAGTA ATGATTACAC TGCTGGAAAC |
| 751 | TATTGGTATG GGTACGCTCC TCATTTGCGA TCAACCACTT CGACAACATC |
| 801 | CTCGCAAAGT GATTCGTTCA ATTCGCAGCA AATACCGCAG CAGCCACAAC |
| 851 | AACAACAGCA GCAGCAGCCA CAGCAACAAA ACAATAGCAA CAATTATTTG |
| 901 | TACTATCAAG GTTTCCAGCA ACCCATTACT AATGCTACGA CTGTTAGTGG |
| 951 | GGCAACGCCA GCTACTTCTA CACTTACGCC CGCTGGAACT GCGTCTGCAT |
| 1001 | CTCTTCTGCA CCAACCTCAA ATTCAGAATC CCGCATCTGC CTCTACAGTG |
| 1051 | CCTCAGCAAA GAATGGTAAT GGGCAATATG GGCTATTATC AGCAACAAGG |
| 1101 | GGCTCCTCAA TATTTTCCTG CACCTCAACA GTCTCTCTTA GGAATCAATG |
| 1151 | ACGTTGATTC AAGAAACCAA ATTGACGATA ACAAACCAAC CCAACCAGGC |
| 1201 | ATTTAA |
| orf19.1219 | |
| (SEQ ID NO: 3) | |
| COORDS: Ca21chr6_C_albicans_SC5314: 879846-881048W Exon(s) | |
| only sequence (1203 nucleotides) | |
| orf19.1219 Length: 1203 Wed Mar. 7 08:44:12 2012 Type: N | |
| Check: 3396 . . . | |
| 1 | ATGGCTAAAC AATACGAGAT TACAGGAGAT TTAGGGTATA ATGGATTCCC |
| 51 | CGTATCTTTA AATTATATCA CCCACTTGCC AGATATATCT ACTTTATACA |
| 101 | ACCCCGAAGT AGTTGTGATT TTCAAATCGT TAATGAAAAG AGACCCCAAA |
| 151 | ACCAAGGAGA AAGCGTTAAA TGATTTATTA ACAGTATCAG CTATTGACGA |
| 201 | CTCCACAATT ATTGCATGGC TTCAAATGTA TCCAAAACTT GCCTTGGATA |
| 251 | ATTCTAGAAG TGTTCGTTTA TTGTCTCATC AAATTCAAGC CAATTTCCTA |
| 301 | AAAATTGTTG GTGGAAAGAC ATATAGCAAA TATTTGAAAA GTTCAATGCC |
| 351 | TATATGGTTA ATGGGGATGT TTGATACAGA TAAATCTGTA TCCTCAATTG |
| 401 | CATATAAAAC ATTATTACAA TCATTTCAAG GAGATAGTTC CAAATTGAAT |
| 451 | AAGACTTGGA GTATTTTTGA AGAACAGATT ATCAATTTGA TTGGGACCAT |
| 501 | TGTCAGTATA GAAACATTGG AAACTTTATC AGACAGAAGA TACACTAGTG |
| 551 | AATCGGAAAT GGTGTCAAAA TATGATCGAG CATTAGTTTG TGGCATCAAT |
| 601 | ATGTTGAGTA AATTGCCAGA TGAAACCATT GTCCCCATAT TAGAAGAAGA |
| 651 | GTCGTTATGG GATCATTTGA GCACCTGTCT CAAAGAAGAT AATATGGATT |
| 701 | TGGTGTTATT CAAAAACTTG TTATTGCTTA TAACCAATTT ATCAGATGAA |
| 751 | AACTTACAAT TGGTCTACAA ACTAGTTTCA AAGAAGTTTA TCAAGATTAA |
| 801 | ATTCAAGTCC AATAAAATAT CCGGAAGTAT AATTTATTCC AATGTGATCA |
| 851 | TACCATTCTG GCAGGCTATA GTACGATTAA CTGAATTTGG CATTAATAAT |
| 901 | AATTTGAAGA AGAATTTCTG GGATCTAGCA GGTAGTAAAA GTTCTACTAG |
| 951 | GTTTTACGAA TACTTGAAAC TTGGTCCATT CAATCTGGAT CCAACTTACT |
| 1001 | ACTCATTGAT TATCAAAGTA TTTCAAGATT TACAGCAGAA GTTAGATGTT |
| 1051 | GTTGATTTTA ACAGCCAGGA GGAATATGAT TATGTGATTA ATTTACTACT |
| 1101 | GAAGCAGTAT TCGACAACTA TGGGATCACT CAAAGCAGGT GCATTAAAAT |
| 1151 | TGTGCATTAA AGACACTGGA TTTATTTACC GTGGAATCAA CTGTATTGAT |
| 1201 | TGA |
| ZCF15 | |
| (SEQ ID NO: 4) | |
| COORDS: Ca21chr4_C_albicans_SC5314: 501568-504402W Exon(s) | |
| only sequence (2835 nucleotides) | |
| orf19.2753\|ZCF15 Length: 2835 Wed Mar. 7 08:46:05 2012 Type: N | |
| Check: 9912 . . . | |
| 1 | ATGGATGCCG ATTTAGAGAA AAGAACTAAA GTATCACGAG CTTGTGATTA |
| 51 | TTGCAAGAAA AGAAAATTCA AATGTTCAGG AGTGTCGCCG TGTGAATTAT |
| 101 | GCACAAAGAA GAATATACAA TGTGAGTTCA GTATTATTGA TCGACGCACA |
| 151 | ATCCGCCGCA AGAATAAGAA AAGAACCATA CAAAATAAAC CAATAACCAA |
| 201 | TACCGTCATA GGATCTCATA AAATTAGTAA AAATGACGAT GATAATGATC |
| 251 | AAATCGATAA ACAAACCTTA AAATTGTTGA CTAAGAAATC TAAAGTACCC |
| 301 | GAACAACTTC AACCGTTGTT AACATTTCCA TTGCACAAAT TAGACAATAA |
| 351 | ACTGAACGAA GAAGAAGAAG AACAAGGGGG AGACAATGAT CACGAAAGGA |
| 401 | GCGAGGAAGG AGAGCACCAA CACAACGACC AATCACTCCC TCCTGGGTCT |
| 451 | AGTTCAGAGT CCCATAATCG TCAGAAAGTG GACATTCAGG GAAACGAGAG |
| 501 | CCACGGTACT AGTGATGGAG ATCTCACCAT CAATGCTACG GGGTTCGATA |
| 551 | TCACAACAAC AAGCAATGAT AGAGTTCACA AAGTTAATCA ACAATCATCA |
| 601 | CCAATGCCAA AGAAAATAGT TATCGCAAGA GAACGTTATC CTCCTAAAGT |
| 651 | ATTATATGAT GGCGAAGGTA ACTTGAGGTA CTTTGGAGAA AGTTCACCAT |
| 701 | TGTCCTTTCT TTTCCAATGT CGGAATGTTT TTGTTGATAA GATCGGGGAT |
| 751 | TCAAATTTCA CACTAGAAAG CAACGATTCC TTGGAAGTCA TTGAAGATTC |
| 801 | TGACGAATCT TATGAAATAA TACAAGTTGC ACTTCCTAGC CGTCAAATAT |
| 851 | TGGATGATAT TATCAAGATT TTCTATATGA ACATTCAACA GGCAGTCTAC |
| 901 | TTTGTAAATA TTGATTTCTT TAAAAGTCAA ATTATTAATC CTGTTTATGA |
| 951 | AAATTATAGC GACTGCACAC CAGGTAAAAT TGCCCTTGTC AATCTAGCCA |
| 1001 | TTGCTGTTGG ATTACTATAC GCTGAAAACA CAAACGATCC ATTAGTGAAC |
| 1051 | CTTTTGCAAT CGCCAACTAT GCAGTCATCA GCTTATTTTG AATTTGGTTT |
| 1101 | CCATTTAGCT AAGAACCATA TGACAAAAGG TGAATTATGG ATAACAGAAG |
| 1151 | CATTTGCCTT GGCATTTTGT TATTACACTT CTAAACAGCA ACGTAATTCT |
| 1201 | GCTTGGCTAA TGTTGGGCAT GGCAATTCGT AATGCACAAG CATTAGGATT |
| 1251 | ACACCGAAAA TTTATAAATG AATCATTTAG AGATCACAAT TATAGGACTC |
| 1301 | ATCGAAGAAG GTTATTTAAA TCCTTATATA TGTTAGACCG TATTAATTCT |
| 1351 | GTATTACTTG GTCGTCCATT GATCATTGAT GATTATGATT GGGATGATTT |
| 1401 | CGATAGTGAA GATATTTACG ATCTTGATGC TGAAGGAAAC CCAATAAAAG |
| 1451 | ATCCACGATT TGAATGCATA ATACAAGCGT GCAAAATATC AAAATTAGCT |
| 1501 | GGGAAGGTGA TACGAAACTA TTATTTAGAT GGAATAATCA ATGCCTATAA |
| 1551 | AGCAGAAAAA TTGGCAATAG AATTGAAGTT GTGGTCACTC AGTTTACCTG |
| 1601 | AACCGCTTCA AATTGATAAA ATCTTTAGGC CAGAAACAGT CAATAAAATG |
| 1651 | CCATTATTAA CTATGCACTT GTCACAATTA TATTCAATTG TATTATTATG |
| 1701 | TCGACCATTT TTCATGTTTG TTATATTGAA TAAAAAACAC AAAAAGCATT |
| 1751 | CCACCAAGAA AGCTAATGGA GAAAATTTAC AAAAGAGACC CAAAACAAGA |
| 1801 | TTAGAAACGT CAATGTGTAA TTTTTGCAAG GCAACCACCA AGTCATCTAC |
| 1851 | TTTGATTATT CAGATGGTTG AAAATTACAT AATCTCAACT AATAGGTTGA |
| 1901 | AGTCAGCAAG AGCAGAACTG AATGGGTTGA TACATACTTG TTTAATGGCT |
| 1951 | AGTTTAATTG TTGGCTTATC GATTTTATTT CTTGAAAGTA ATGGATATAG |
| 2001 | TATTGATGAT GGATACTCAT CGAGTCAGTT AATGAAATAT TTGAATTCAT |
| 2051 | CAAAACAGAT TTTCCAATGT TTCGATAGAC TGAAAAATGC ATTATCAATG |
| 2101 | AAATTTCATA ATATAATTGA ACAAATGCAA TTAGCATTGA TGAATAAATT |
| 2151 | CAATCTTGAT TACAATGGTG ATAAAATTAT TAATTCTCAG GAGAGTCAAA |
| 2201 | ACCAACAAAG ACAACATGGA CATGATACAT TACCACAAGC ACAACAAGCA |
| 2251 | CCACCAGCGG TACCACCACC ACCGCCACAT CAACAAACAC AACCTAGTAA |
| 2301 | CAACATGACA GCAAATTTTA ACTTCAATTA TAATAACAAT GATAACGCCA |
| 2351 | CTATGAAAGC GGAAATGTTA ACA7ACCAAC AAACTTCATT TAATGAAGAT |
| 2401 | TATGATAAAT TTATTGATAC ATTTGGTAAT TTGCCCAACT TTTATTCTTC |
| 2451 | TAATCATAAT TATGGCAACA AATCTAATTC AAGCAATGAT TCCCATCAAT |
| 2501 | ATCAAACTAA TGGTGTTGAT TATTATAATG CCAACAATTA TCACAACAAC |
| 2551 | CAAACCCCCA ACCACAACCA CAACCCCAAT CACAACCACA GTCACAGTCA |
| 2601 | CAGTCACAGT AGCAGCAATG TACCACAGTA TGATGGCAAT AATTATAACA |
| 2651 | ACCACTTGAC GAACCATCTG CCAGAATCCG TAACTAGTTC CACGAATTTA |
| 2701 | TCTACATCAA GTAGTAATTT TTCAAATCTG ACGTATCATC AAGATCCAAA |
| 2751 | TAAGAATAAT TTGACAGATT CATTAGATGT ATTTATGTAT AATGTTGAAT |
| 2801 | TGAGTGATAT TTTATATGAT GTGAAACCCA ATTAA |
| SAP8 | |
| (SEQ ID NO: 5) | |
| COORDS: Ca21chr3_C_albicans_SC5314: 549451-548234C Exon(s) | |
| only sequence (1218 nucleotides) | |
| orf19.242\|SAP8 Length: 1218 Wed Mar. 7 08:49:11 2012 Type: N | |
| Check: 6353 . . . | |
| 1 | ATGGTCTCCA TTATTACTTT TACCAAAAAC GTTCTTGTTA CACTTGCTTT |
| 51 | TGCTTTATTG GCTCAAGGTC TTGCTATCCC TGAAGACATT GATAAAAGAG |
| 101 | CTGAAAAAGT TGTCTCATTA GATTTTACCG TTACCAGAAA ACCTTTTAAT |
| 151 | GCTACTGCTC ATGGACAACA TCATCAATCC CAACAGCAGC AGCAACAACA |
| 201 | ACAACAACAA CCAGCTCAAA AAAGAGGAAC TGTTCAAACA AGTTTGATTA |
| 251 | ATGAAGGTCC ATCATATGCT GCTACCATCA CTGTTGGTTC AAACAAACAA |
| 301 | CAACAAACTG TTATTGTTGA CACAGGTTCT TCTGATTTAT GGGTGGTTGA |
| 351 | TTCTGCTGCC GTTTGTCAAG TCACTTATCC TGGTCAATCA CCAACTTTTT |
| 401 | GTAAACAAGA TGGTACTTAT AAACCTTCTT CTTCCACAAC ATCTCAAAAT |
| 451 | TTAGGTAAAG CTTTCTCCAT TAGATATGAA GATGGAAGTT CTTCTCAAGG |
| 501 | TACTGTTTAT AAGGATACTG TTGGTTTAGG TGGTGCTTCA ATTACCAATC |
| 551 | AACAATTTGC TGATGTCACT ACGACTTCAG TTGATCAAGG TATTTTAGGT |
| 601 | ATTGGATTCA CTGGTGATGA AAGTAGTCCA ACCTATGATA ATGTTCCTGT |
| 651 | CACTTTGAAA AAACAAGGAA TTATCAACAA GAATGCTTAT TCTTTATATT |
| 701 | TGAATTCTGC TTCTGCTTCA TCTGGTACCA TTATTTTTGG TGGGGTTGAT |
| 751 | AATGCTAAAT ACACTGGTAG TTTGACCGCA TTACCAATCA CTTCATCCAA |
| 801 | TGAACTAAGA GTTCAATTGA GTACTATTAA CATTGCTGGA ACCACTGTTA |
| 851 | GTGCTTCAAC TACCCCAGTT TTGGATTCAG GTACTACTCT TACTTATTTT |
| 901 | TCTCAAACTA TTGCTGATAA ATTGGCTGCT GCCGTTGGTG CCAAATGGAA |
| 951 | TAGTTATTAT CAACTTTATA CTTCTAGTTG TAATCTTGCT GGAAATATTG |
| 1001 | TTTTCAATTT TGCTAAAGGG GTTACTATTT CGGTTCCATT ATCAGAATTT |
| 1051 | GTTCTTCAAG ATGGTAATTC TTGTTATTTT GGTGTTAGTA GAGATCTGGC |
| 1101 | CACTATTTTG GGGGATAATT TCTTGAGAAG AGCTTATGCA GTTTATGATC |
| 1151 | TTGATGGGAA CACCATTCTG TTGGCTCAAG TCAAATACAC TACTTCTTCA |
| 1201 | AGTATTTCTA CTTTATAG |
| CMP1 | |
| (SEQ ID NO: 6) | |
| COORDS: Ca21chr1_C_albicans_SC5314: 128619-126790C Exon(s) | |
| only sequence (1830 nucleotides) | |
| orf19.6033\|CMP1 Length: 1830 Wed Mar. 7 08:50:35 2012 Type: N | |
| Check: 555 . . . | |
| 1 | ATGTCAGGAA ATACTGTTCA ACGTAATACT GAACAAATCA ATAATGCATT |
| 51 | AAATGCAATT CAACATAGAC GAACCACCAC AGGTAATGAA CTGAATACCA |
| 101 | ATACTAATCA ACGACAAATC TTGCAAAATC CCACTGGTAG AGATTATACT |
| 151 | ATTTATGTTA CAGATGATGG TGAAAAATAT TCAACGGTTG AAAGAGCTGT |
| 201 | AAAATCAGTT GATCCACCAG CAACATTTAA ACCTAAAGAT GAACAAGTTT |
| 251 | TTTATCCTAA TGGTAAACCA AATCATCAAT TTTTAAAACA ACATTTTATT |
| 301 | CATGAAGGTA GATTACATGA ACATCAAGCC ATTCAAATTT TAAAACAAGC |
| 351 | AACTCATTTA TTAAGTAAAG AACCAAATTT ATTAAGTGTT CCGGCACCAG |
| 401 | TAACTATATG TGGAGATGTT CATGGTCAAT ATTATGATTT GATGAAATTA |
| 451 | TTTGAAGTTG GTGGTGATCC CGCATCTACT AAATATTTAT TTTTGGGTGA |
| 501 | TTATGTTGAT CGAGGTTCAT TTTCTATTGA ATGTTTACTT TATTTATATT |
| 551 | CATTGAAAAT TAATTATCCT GATACATTTT GGATGCTTAG AGGTAATCAT |
| 601 | GAATGTCGTC ATTTAACTGA ATATTTTACA TTTAAAAATG AATGTTTACA |
| 651 | TAAATATTCT GAAGAATTAT ATGAAGAATG TCTTGTTAGT TTTAATGCTT |
| 701 | TACCATTAGC AGCAATTATG AATGAACAAT TCTTTTGTGT TCATGGAGGA |
| 751 | TTATCACCTC AATTGACCAG TTTGGATAGT CTTAGAAAAT TACATAGATT |
| 801 | TAGAGAACCT CCGACCAAGG GGTTAATGTG TGATTTATTA TGGGCTGATC |
| 851 | CAATTGAAGA ATATGATGAT GATAATCTTG ATCAAGAATA TGTCACTAAT |
| 901 | GTGGTTAGAG GTTGTTCATT TGCCTTCACT TATAAAGCTG CTTGTAAATT |
| 951 | TTTGGATAGA ACAAAATTGT TATCAGTGAT TAGAGCTCAT GAAGCTCAAA |
| 1001 | ATGCTGGTTA TCGAATGTAT AAGAGAACTA AAACAATGGG GTTCCCGTCA |
| 1051 | TTATTAACCA TGTTTAGTGC TCCGAATTAT TTGGATAGTT ATAATAACAA |
| 1101 | AGCGGCGGTT TTAAAATATG AAAATAATGT GATGAATATT AGACAATTTA |
| 1151 | ATGCTTCACC TCATCCTTAT TGGTTACCTC ATTTCATGGA TGTTTTCACT |
| 1201 | TGGTCATTAC CGTTTGTGGG TGAAAAAGTT ACCGATATGT TGGTATCTAT |
| 1251 | TTTAAATGTT TGTACTGAAG AAGAATTGGA TGAAGATTTA CCATTTAGTG |
| 1301 | AAGCTGAAAT AGGTGTGACC CCAACTACAA CTACAACAGG AGCAACACCA |
| 1351 | GTATCACCAA AAGCTTATCC ACCATCAAGT CGTATTACCT CACCTCACAA |
| 1401 | ATCAACCAAA CTTGTTGAAT CAAGAGCCAA CGAAGAAGAG AAGGCTAATG |
| 1451 | ATGGTGATGA TGACTCTGAA ATGACATTAG AAGAAAAGAA ACAAGCTTTA |
| 1501 | CGTAACAAAA TCATTGCTAT TGGTAAAATG TCAAGGATGT TCCAAGTTTT |
| 1551 | ACGAGAAGAA CAAGAAAATG TTGCTCATTT AAAAGAATTA AATCGGGGAT |
| 1601 | CATTACCCAA AGGTTCATTA TTACATGGTG CTGATGGATT AAAAAATACT |
| 1651 | ATTAATTCAT TTGAAGAAGC TAAAGCTGCT GATAGAGTTA ATGAAGCATT |
| 1701 | ACCACCTTCA CCAGAAGATT TGCAACGATT AAAACAAGAA AAAAATACCA |
| 1751 | GAATTAGACA ACAAATTGAA AATCAAGAAA TGAGTGGACC AGTTTTCCAA |
| 1801 | AGATTAATAA GAAGATTATC TCAAAGTTAA |
| IFF11 | |
| (SEQ ID NO: 7) | |
| COORDS: Ca21chr3_C_albicans_SC5314: 95651-97186W Exon(s) | |
| only sequence (1536 nucleotides) | |
| orf19.5399\|IFF11 Length: 1536 Wed Mar. 7 08:51:54 2012 Type: N | |
| Check: 2491 . . . | |
| 1 | ATGTTATTGT CAAACCTTGT TATTCCCTTA TTAGCTACCA GTGCTACTGT |
| 51 | GTCAGCTACA TTTGATGTCA CTTCTTCATT AACCAAATAT GCTGCATTTG |
| 101 | ATTTTGGTGT TGGTGACATT TCCATTCAAA AGAGTGCTTC ATTTTCTTTA |
| 151 | ATCAACACTT TTGATTGTGC TTTCCAAGGT GATATTAACA TCGAAAAAGA |
| 201 | TTGTGAGTTC CTTATTGCTT CTACTGCTAA AGCCTTAAAA CTTACCTTTA |
| 251 | ACAACATTTT CCACAAGATT GAAAACAAGG GTAAATGGAT CGTTCACACC |
| 301 | TTAGAGTCTG CCGCTGCTAG TACATGTAAT ATTATTGTCA AGTCTTTTGT |
| 351 | CAACACTGGT GAAATCATTT TGGCTTTCAA AGGTCACGTT TTGGCTCCGA |
| 401 | TCATTAACAT TGCCGCTTCC CATTGGGTAA ATCACGGTTG TTTACATCTT |
| 451 | TTTCAAGAAA TCAAATCAAT CTCTGTCGCC ATTTTGGGTG TTGTTGGTGG |
| 501 | CACAATTGAA AACCATGGTA CTATTTGTTT AACCAACCAG CTCTGTAAAC |
| 551 | AAGTAACAAA AATTATTGGT AGTGGGTGTA TTGCCTTGGA AAAATCTTCT |
| 601 | TCATTCTTTA TTTCCAACTC ATTCTTGTCA ATTGATGCTC AACATACTTT |
| 651 | CTACTTGGGT GAAGGTAACC CAACTATTCA AGCTCAAGCA GTGTCACTCC |
| 701 | CACA7ACATT CAAGGTTGCC AATTTTGGTG CTAATGGTTC TCATAAAATT |
| 751 | GGTTTGAACT TGCCTTTGTT GTCTATTACT ATTGCTGGGA AAAAAGGTTG |
| 801 | GTCTTACGAC ACCAAAACTG GTATTTTGAC ATTGACTGCT AATGGATTCA |
| 851 | TTTCCCAAAA GTTTGACATT GGTCTTGGTT ACGATCCTTC TAAGTTTGAA |
| 901 | GTTTGTACTG ACAATTCAGT TGGTATTGTT TCCGTTATCA AAGGTTCTAT |
| 951 | CAAATACAAT GGTCCATGTC CACATGCTGG AAGACCATCA GTGTGTCAAA |
| 1001 | TTTGTCCAGG CGTTCCAACT CCTCCAGTTA TTACATCAAG TGCCACCGCT |
| 1051 | ATCACAAGCA CCCAATCA7A AACTACTAGT TCCTCATCCA CTTCAAGTAT |
| 1101 | TGCTACTACT AGTACTACCA GAAGGTAACC TAAGCCTACT ACTGTTTCTA |
| 1151 | CTAGCTCTAT TGCTTCTACA ACTACTCCAG ATGGATCTTA CTCCACCTCA |
| 1201 | TTCGTCACTG TTACTGCTGA AACCACTAAG GTTGTTACTA TCACTTCTTG |
| 1251 | TTCTAACAAT GCTTGTCATC CAACAACTGC TCCAACTGGT CTTACTGTCG |
| 1301 | TCACACTTAC TACTTCTGAT GTCAAAACTG TTGTCACTAC TTACTGTCCA |
| 1351 | TTGACTCAAA CTGTTACTTT GGGTGTTACC GGTACCAAGA CTGTCGATTT |
| 1401 | GGGTTGCCCA ACCGGTTACT TTGGTTACAA CGGGTACATT GGTAAAGGTG |
| 1451 | TTAGTATCGG TCATGGCGGT GCTGCTGCCA GTGCCAGTGC TGGTTTCCAA |
| 1501 | ATTGGATTTG ATATCTTCAA AAATCACCAC TTTTAA |
| CAP1 | |
| (SEQ ID NO: 8) | |
| COORDS: Ca21chr3_C_albicans_SC5314: 479014-480513W Exon(s) | |
| only sequence (1500 nucleotides) | |
| orf19.1623\|CAP1 Length: 1500 Wed Mar. 7 08:54:05 2012 Type: N | |
| Check: 3271 . . . | |
| 1 | ATGACAGATA TTAAAAGAAA TTTTTCAGAT ATTGCCTCAC CAGCAAATCT |
| 51 | AGATGATACC AAGAAACTTC ACGTAGATTC TACTGCAACA ACAAAAGTGG |
| 101 | GCAGAAAACC AATCGATACC GAACCTAAAT CAAAGAGAAC TGCTCAAAAT |
| 151 | AGAGCTGCTC AAAGGGCATA CCGTGAACGT AAAGAACGTA AAATGAAAGA |
| 201 | GTTGGAAGAT AAGGTAAGAT TATTAGAAGA TGCAAATGTC AGAGCCCTAA |
| 251 | CTGAAACCGA TTTTTTACGA GCTCAAGTAG ATGTGTTAAA AAATGAGTTG |
| 301 | GCTAAATATA CTGGTGGTAG CGATTTTCTG GATTTGAATT TACCTACTAA |
| 351 | AGTTGGTCAT TTATCACATC CAAATAATCA TCACAGTAAT GTTTCTACCG |
| 401 | GTACACCTCA TGGTTCAATA TCATCTTCTA ATTCTGTTGC AAGTTTAGAT |
| 451 | AATGACAAAC CTTCTAGTGC TTCATCGGTA TCAAACAATT CACCTGGTTT |
| 501 | TGCTTTTGAT AATCCTTGGT CTAAAGATAA CATACAAAAG TTAAAGCACC |
| 551 | AACATCAACA ACAACAACAA AAGGTTCCCC AAGGTGTGCC GGATTTGGTG |
| 601 | CTGGGTTCAT CTTCATCGTC TACACCTTTA AATGACAATT TATTGGTCAC |
| 651 | TCCTGAATCG TTGACTGGTT TATCTACATC TAGTAAATAT ACTGGCCAGA |
| 701 | ATAATGTACC AACCAATTTG GATTTCACCA ATCAGTTTGA TGAACAAGTT |
| 751 | GATCCATTTT GTGTTAAATT GAATGAAGCA TGCGGAACCA AAAGTAACCC |
| 801 | AGTGCCCAAA TTTAAACGTT CAGGTTCTAA AGCAAATACT TCAGTAACAA |
| 851 | ATAATTCCCC ACTTGCCCAC TTGGTATCAC CGGAATCTCA ACAATATACC |
| 901 | AATTCTTCTA ATATCGACTT TATGAATGAT CCATTTTTCA ATGGTGTGGG |
| 951 | AACTGATTAT AACTTTAATT TCGATAGCAA GAATGGTTCC ATTCAAGATC |
| 1001 | CTTTATCGTT TCTTCAGGAT GACAACTTTG ATCTTGCATT GGCATTTGGT |
| 1051 | GATCCAAGTC CTACTGGTAA TGAAGCAGAA GCCGATCCAA TTTCATTATT |
| 1101 | AACAACAGAA GAATCTATAT ATGATCCATT GACAAACAAC AGTGATAAAC |
| 1151 | TTTGTAGTAC GGTTAAAGCT GATGATGTTA ATACTGACTT CAATTTCAAT |
| 1201 | GATTTTGTCA AAAATTCATT ACCTGAAAAA CAAGAGAAAG GTAAATATGA |
| 1251 | ACCACCATCA ACATCAAAGA CTACAAATAA TAATGAAGAA GAAGATAAAG |
| 1301 | ATGAAGTTGT GCCGGCACCT CCACAAACAC TCAAATGTAG TGAAATTTGG |
| 1351 | GATAGAATAA CATCACATCC AAAATATACT GAACTTGATA TTGATGGGTT |
| 1401 | GTGTAATGAG TTGAAAAGTA AAGCTAAATG TTCTGAAAAG GGAGTAGTGA |
| 1451 | TAAATACTGC TGATGTGAAT CAATTACTAG AGCGAAGTAT AAAACATTAA |

1. A multi-organism model system comprising, in co-culture, the nematode Caenorhabditis elegans (C. elegans), a fungal pathogen that infects humans, and a source of food for the C. elegans.

2. The multi-organism model system of claim 1, wherein the C. elegans is a wild type C. elegans and the pathogen comprises a mutant gene.

3. The multi-organism model system of claim 2, wherein the wild type correlate of the mutant gene encodes a protein that increases the pathogen's resistance to reactive oxygen species.

4. The multi-organism model system of claim 1, wherein the C. elegans is a mutant C. elegans and the pathogen is a wild type pathogen.

5. The multi-organism model system of claim 4, wherein the wild type correlate of the gene that is mutant in the mutant C. elegans encodes a protein that facilitates the production of reactive oxygen species in C. elegans.

6. The multi-organism model system of claim 1, wherein the source of food is a heterologous cell.

7. The multi-organism model system of claim 6, wherein the heterologous cell is a bacterial cell, optionally engineered to express a potential anti-fungal agent or an agent that inhibits gene expression in C. elegans.

8. The multi-organism model system of claim 1, wherein the co-culture is configured in a multi-well plate and/or configured for high throughput screening.

9. The multi-organism model system of claim 1, further comprising a potential antifungal agent.

10. (canceled)

11. The multi-organism model system of claim 1, wherein the fungal pathogen is of the genus Candida.

12. (canceled)

13. The multi-organism model system of claim 1, wherein the system further comprises an agent that inhibits the expression of a gene or the activity of a gene product in the C. elegans or the human pathogen.

14-15. (canceled)

16. The multi-organism model system of claim 1, wherein the fungal pathogen comprises a mutation in one or more of the genes CMP I, IFF II, SAP 8, DOT4, orfl9.6713, orfl9.1219, and ZCF15 or a homolog or homologs thereof.

17-19. (canceled)

20. A method of identifying a host factor that promotes fungal pathogenesis, the method comprising:
(a) providing a co-culture comprising a C. elegans comprising a mutant gene, a wild type fungal pathogen that infects humans, and, optionally, a source of food for the C. elegans; and
(b) carrying out an assay to determine whether the fungal pathogen infects the C. elegans, wherein inhibition of the expected infection level identifies the protein encoded by the wild type correlate of the mutant gene as a host factor that promotes fungal pathogenesis.

21. (canceled)

22. A method of identifying a gene in a fungal pathogen that helps protect the pathogen from reactive oxygen species, the method comprising:
(a) providing, in co-culture, a mutant host organism that is deficient in the production of reactive oxygen species and a fungal pathogen comprising a mutation; and
(b) carrying out an assay to determine the extent to which the mutant host organism is infected by the fungal pathogen, wherein a degree of infection that is greater than the degree of infection observed when the pathogen is exposed to a wild type host organism indicates that the mutation is in a gene that helps protect the fungal pathogen from reactive oxygen species.

23. The method of claim 22, further comprising step (c): exposing the gene that protects the fungal pathogen from reactive oxygen species to a potential anti-fungal agent.

24. The method of claim 23, further comprising step (d): determining whether the potential anti-fungal agent reduces the expression of the gene sequence or inhibits a protein encoded by the gene sequence, wherein an agent that reduces the expression of the gene sequence or inhibits a protein encoded by the gene sequence, is a putative anti-fungal agent.

25. (canceled)

26. The method of claim 22, further comprising a source of food for the C. elegans.

27. The method of claim 26, wherein the source of food is E. coli.

28. The method of claim 22, wherein the fungal pathogen is of the genus Candida.

29-32. (canceled)

33. The method of claim 23, wherein the potential anti-fungal agent is a nucleic acid sequence and exposing the gene that protects the fungal pathogen from reactive oxygen species to the potential anti-fungal agent comprises co-culturing the fungal pathogen and a heterologous cell that expresses the nucleic acid sequence.

34-40. (canceled)
